(19) United States
(12) Patent Application Publication
(10) Pub. No.: US 2015/0338403 A1
(43) Pub. Date: Nov. 26, 2015

(54) EMBODIMENTS OF A COMPOSITION COMPRISING DIATOM FRUSTULES AND A METHOD OF USING

(71) Applicant: Oregon State University, Corvallis, OR (US)

(72) Inventors: Alan X. Wang, Corvallis, OR (US); Gregory L. Rorrer, Corvallis, OR (US)

(73) Assignee: Oregon State University, Corvallis, OR (US)

(21) Appl. No.: 14/811,639

(22) Filed: Jul. 28, 2015

Related U.S. Application Data
(63) Continuation of application No. PCT/US2014/014286, filed on Jan. 31, 2014.
(60) Provisional application No. 61/759,833, filed on Feb. 1, 2013.

(51) Int. Cl. G01N 33/553; G01N 21/65

(52) U.S. Cl. CPC G01N 33/553; G01N 21/658; G01N 2201/068

(57) ABSTRACT

A composition comprising a diatom frustule and a metal coating, and a method for making the same, are disclosed herein. The metal coating may comprise a metal film or metal nanoparticles, which may be attached to the surface via a linker. The composition has a surface coverage ratio of from about 1% to about 100%. The composition may also comprise an antibody. Also disclosed is a method for using the composition comprising contacting the composition with a target molecule, exposing the composition to light, and measuring the resulting Raman scattering.

### CROSS REFERENCE TO RELATED APPLICATION

This application is a continuation of International Application No. PCT/US2014/014286, filed Jan. 31, 2014, which claims the benefit of the earlier filing date of U.S. Provisional Application No. 61/759,833, filed Feb. 1, 2013, both which are incorporated herein by reference in their entireties.

### ACKNOWLEDGMENT OF GOVERNMENT SUPPORT

This invention was made with government support under grant number 1240488 awarded by the National Science Foundation (NSF) through the Emerging Frontiers in Research and Innovation (EFRI) program, and under grant number 9R42ES024023-02 awarded by the National Institute of Health. The government has certain rights in the invention.

### FIELD This invention concerns a composition comprising a diatom frustule and a metal coating as a substrate for surface-enhanced Raman scattering.

### BACKGROUND

Surface-enhanced Raman spectroscopy (SERS) has been widely investigated as an analytical tool for detecting various biological and chemical molecules with single molecular sensitivity due to the strong electric fields induced by plasmonic resonances. Even though an enhancement factor as large as 10¹⁴ has been reported by comparing the measured SERS cross-section of a molecule in the hot-spot to a typical Raman scattering cross-section, controlling the location and density of such hot-spots remains a major challenge for reliable SERS sensing. Moreover, increasing the average enhancement factor of the whole SERS substrate is even more desirable than obtaining a few extremely strong but very rare hot spots, as the former device can increase the detection probability while reducing the excitation laser power and the integration time for high-throughput optical sensing applications. It has been theoretically proved and experimentally confirmed that placing metallic nanoparticles near or inside dielectric microcavities can form hybrid photonic-plasmonic modes, which increases the quality-factors and the local electric field for SERS. This concept has been successfully applied to SERS sensing by decorating dielectric ring resonators and photonic crystals with metallic nanoparticles. In these hybrid nanostructures, the guided-mode resonances (GMRs) of the photonic crystal slab efficiently couple to the localized surface plasmons (LSPs) of the metallic nanostructures, resulting in a higher local electric field to enhance the SERS signals. However, such artificial photonic crystals require top-down fabrication techniques, such as optical lithography and reactive-ion etching (RIE), which are costly and complex for particular applications such as point-of-care and disposable sensors.

### SUMMARY

In view of the above, there is a need for an inexpensive source for photonic crystal-like structures that can be used for applications such as SERS sensing. Disclosed composition embodiments comprising a diatom frustule and a metal coating that at least partially covers the frustule surface address that need. The metal coating may comprise a metal film, or may comprise metal nanoparticles. As used herein the term “nanoparticles” includes nanoparticles, nanowires, nanorods or any combination thereof. In some embodiments the nanoparticles are silver nanoparticles.

The composition may further comprise a linker, which, in turn, may comprise an amine group. In certain working embodiments, the linker is aminopropyltriethoxy silane or aminopropyltrimethoxy silane.

The composition has a surface coverage ratio. In certain embodiments, the surface coverage ratio is from about 1% to about 100%, and in particular embodiments is about 50%.

In some embodiments the composition may also comprise an antibody.

Also disclosed herein are embodiments of a method for making the composition, comprising providing a diatom frustule comprising a surface, and contacting the surface with metal to form the metal coating on at least a portion of the surface. In some embodiments, the method further comprises contacting the surface with a linker, which may comprise an amine group. In certain embodiments, the method further comprises contacting the composition with an antibody.

In particular embodiments, the method comprises providing a diatom frustule comprising a surface, contacting the surface with a linker, and contacting the linker with metal nanoparticles to form a metal coating on at least a portion of the surface. The method may further comprise contacting the linker with a crosslinker compound, and contacting the crosslinker compound with an antibody.

Also disclosed herein are embodiments of a method for using the composition, comprising contacting the composition with a target molecule, exposing the composition to light comprising a particular wavelength, and obtaining a spectrum. In some embodiments the particular wavelength is a resonance wavelength of the target molecule, and in other embodiments it is a non-resonance wavelength. In particular examples, the composition comprises nanoparticles, typically silver nanoparticles, and may further comprise a linker, and/or an antibody. In certain examples, the metal coating comprises a first nanoparticle, the composition includes a first antibody, and the method further comprises contacting the target molecule with a second antibody attached to a second nanoparticle.

In some embodiments the obtained spectrum is a Raman spectrum, and in certain embodiments it is a SERS spectrum. In particular working embodiments the spectrum comprises at least one signal, which has at least a 2-fold enhancement, such as a 2to 10-fold enhancement, typically a 2- to 6-fold enhancement, compared to a signal from a spectrum obtained without a diatom frustule.

Also disclosed is a sensor comprising a diatom frustule comprising a surface, a linker connecting a metal nanoparticle to the surface, and an antibody. In certain embodiments the sensor further comprises a second metal nanoparticle and a second antibody.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides a representative SEM image illustrating the nanoparticle coverage when the nanoparticle solution is diluted with water in a ratio of 1:10.

FIG. 2 provides a representative SEM image illustrating the nanoparticle coverage when the nanoparticle solution is not diluted.

FIG. 3 provides a representative SEM image of Ag nanoparticles on a diatom frustule.

FIG. 4 provides a representative SEM image of Ag nanoparticles on a glass substrate.

FIG. 5 is a schematic drawing illustrating one embodiment of an immunosensing method for diatom-SERS sensors.

FIG. 6 provides a representative SEM image of a diatom frustule.

FIG. 7 provides a representative SEM image of Ag nanoparticles self-assembled on a diatom frustule.

FIG. 8 provides a dark-field image of self-assembled Ag nanoparticles on a diatom frustule and on a glass substrate.

FIG. 9 provides SERS spectra of 1 μM Rhodmine 6G (R6G) coated on Ag nanoparticles-on-diatom and Ag nanoparticles-on-glass, under resonance conditions using an excitation wavelength of 532 nm.

FIG. 10 is a graph of Raman signal intensity versus concentration, indicating the intensities of the Raman band at 1368 cm⁻¹ for different R6G concentrations, under resonance conditions using an excitation wavelength of 532 nm.

FIG. 11 provides SERS spectra of 100 μM R6G coated on Ag nanoparticles-on-diatom and Ag nanoparticles-on-glass, under non-resonance conditions using an excitation wavelength of 785 nm.

FIG. 12 is a graph of Raman signal intensity versus concentration, indicating the intensities of the Raman band at 1368 cm⁻¹ for different R6G concentrations, under non-resonance conditions using an excitation wavelength of 785 nm.

FIG. 13 provides an optical image of the area scanned for SERS measurement.

FIG. 14 provides a map of Raman signal intensity at 1368 cm⁻¹ of the area indicated in FIG. 13, with an integration time of 60 seconds, a grating groove density of 300/mm and an excitation power of 2.1 mW.

FIG. 15 provides a TEM image of a diatom frustule with 2-D periodic pores, and a schematic diagram of a diatom photonic crystal slab with unit cell parameters based on the TEM image.

FIG. 16 provides transmission spectra of diatom frustules with different numbers of unit cells illuminated by a 5 μm normally incident Gaussian beam.

FIG. 17 provides transmission spectra of diatom frustules of a 10×10 unit cell frustule illuminated by a Gaussian beam with different beam widths.

FIG. 18 provides peak electric field enhancement of a diatom frustule illuminated by Gaussian beams with different widths, and the electric field distribution in the middle plane and on top of the diatom frustule.

FIG. 19 is a graph of peak electric field enhancement versus wavelength of a 50 nm Ag nanoparticle at different locations on a diatom frustule, and schematic diagrams indicating the locations.

FIG. 20 is a graph of peak electric field enhancement versus wavelength of a 50 nm Ag nanoparticle dimer (25 nm nanoparticles at 2 nm gap size) at different locations on a diatom surface, and schematic diagrams indicating the locations.

FIG. 21 provides a field enhancement profile of different shapes of metal nanostructures on a diatom at 480 nm (log 10 scale)

FIG. 22 provides the normalized field enhancement obtained by monitoring the individual metal structures of FIG. 21 located at the hotspots.

FIG. 23 is a graph of normalized transmission versus wavelength of different shapes of metal structures under three different conditions: on a diatom substrate; in free space; and on a glass substrate.

FIG. 24 provides SEM images of diatom frustule samples comprising evaporated silver films, with and without thermal annealing, and an image of a diatom frustule comprising self-assembled nanoparticles.

FIG. 25 provides SERS spectra from R6G molecules adsorbed on self-assembled Ag nanoparticles on diatom frustules, with an acquisition time of 1 second.

FIG. 26 provides SERS spectra from R6G adsorbed on an annealed Ag thin film on a diatom frustule.

FIG. 27 provides SERS spectra from R6G molecules adsorbed on self-assembled Ag nanoparticles on a glass substrate, with an acquisition time of 1 second.

FIG. 28 provides SERS spectra from R6G adsorbed on an annealed Ag thin film on a glass substrate.

FIG. 29 provides a comparison of Raman signal intensities at 1364.3 cm⁻¹ peak between diatom frustules and bare glass substrates, with signals observed from self-assembled Ag nanoparticles.

FIG. 30 provides a comparison of Raman signal intensities at 1364.3 cm⁻¹ peak between diatom frustules and bare glass substrates, with signals observed from annealed Ag thin films.

FIG. 31 provides SERS spectra of antigen-antibody binding.

FIG. 32 is a schematic diagram illustrating SERS immunoassay including multiple formats for detection: (A) illustrates an Au nanoparticle core coated with a shell into which extrinsic Raman labels and antibodies are immobilized; (B) illustrates an Au-coated Ag nanoparticle probe conjugated to both extrinsic Raman labels and capture antibodies; and (C) illustrates antibodies conjugated to an Au nanoparticle using extrinsic Raman labels.

FIG. 33 is a graph of extinction ratio versus wavelength of an unmodified diatom, Ag nanoparticles on glass and nanoparticles on a diatom frustule.

FIG. 34 provides Raman spectra of 5 mM R6G on a diatom frustule and on a glass substrate, with the excitation power of 1.2 mW and an integration time of 5 seconds.

FIG. 35 provides SERS spectra on a diatom frustule and on a glass substrate, with the inset showing where the SERS signals were collected.

FIG. 36 is a schematic diagram illustrating one embodiment of a method for making and using a composition comprising a diatom frustule with self-assembled nanoparticles for an immunoassay.

FIG. 37 provides an SEM image of diatom frustules with self-assembled Ag nanoparticles and nanowires.

FIG. 38 provides an expanded view of the diatom frustules of FIG. 37 showing morphologies of Ag nanoparticles and nanowires and sub-pores of the frustules.

FIG. 39 is a graph of Raman intensity versus Raman shift, illustrating the average SERS spectra of pure DTNB and DTNB-labeled diatom frustules in an immunoassay.

FIG. 40 is a bright field microscope image of a single diatom frustule challenged by mouse IgG in an immunoassay using an anti-mouse IgG antibody.

FIG. 41 is a bright field microscope image of a single diatom frustule challenged by human IgG in an immunoassay using an anti-mouse IgG antibody.

FIG. 42 is a Raman mapping image of a single diatom frustule challenged by mouse IgG in an immunoassay using an anti-mouse IgG antibody.

FIG. 43 is a Raman mapping image of a single diatom frustule challenged by human IgG in an immunoassay using an anti-mouse IgG antibody.

### DETAILED DESCRIPTION

### I. Overview

Certain disclosed embodiments concern a new surface-enhanced Raman scattering (SERS) substrate to enhance the sensitivity for biomolecular and chemical detection. SERS has demonstrated single-molecule detection capability and is becoming intensively investigated due to its significant potential in chemical and medical applications. The SERS applications for biomolecule detection, however, have been restricted by the difficulty associated with fabricating ultrasensitive and reproducible surface-plasmonic-resonance (SPR) substrates. For certain disclosed embodiments, single-celled algae, called diatoms, are used as biologically fabricated SERS substrates. A thin layer of a substrate metal, such as a gold film, a silver film, or a gold/silver film is evaporated onto the diatom. Alternatively, the diatoms are coated or at least substantially coated with high-density nanoparticles by self-assembling. Compared with nanoparticles on flat glass substrates, the average sensitivity of disclosed SERS substrates can be increased by a factor 10×, and the single-molecule detection capability can be increased by a factor of 100×.

One aspect of certain disclosed embodiments is that a diatom frustule can serve as a platform to assemble plasmonic nanoparticles. Diatom cells make silica shells called frustules. Frustules have periodic structures that are ordered at the micro- and nanoscale, which provide a cost-effective and scalable approach to obtain photonic crystal structures. Diatom frustules that comprise plasmonic nanostructures not only enhance the surface plasmons through the photonic crystal resonance, but also preserve fine-patterned (sub-100 nm) pore features of the frustules for assembling plasmonic nanoparticles. Hybrid plasmonic-dielectric surfaces with 3-D morphologies provide strongly localized electric fields, which increases the sensitivity of SERS. The other significant merit comes from the surface of the diatom frustule. Compared with glass substrates, diatom frustules are ideal platforms for biosensing due to the porous structure and a surface rich in reactive silanol groups. A surface-functionalization technique has been used for certain SERS substrates to achieve higher sensitivity and better specificity. And, the sub-100 nm pores of the diatom frustules serve as nanofluidic channels, where a biofluidic sample flows through them. The overlap of nanofluidic channels with the hot-spots between metallic nanoparticles facilitates ultra-high sensitivity and detection probability.

One advantage of certain disclosed embodiments is enhanced the sensitivity and specificity of SERS sensing, which can be broadly used for biomedical and chemical applications.

There are different approaches to increasing the sensitivity of SERS substrates, including integrating metallic nanoparticles on man-made photonic crystals. However, the fabrication cost is too high, which prohibits this approach from being widely used. Certain disclosed embodiments use low-cost, biologically assembled diatom frustules to provide a high sensitivity SERS substrate that can be made without using any expensive photolithography and/or ion etching processing.

### II. Diatoms

Diatoms are photosynthetic marine micro-organisms that create their own skeletal shells of hydrated amorphous silica, called frustules. Frustules possess hierarchical, nano-scale, photonic crystal features. Such nano-biosilica is formed by a bottom-up approach at ambient temperature and pressure when a diatom takes up water-soluble silicic acid from the environment. The silicic acid precipitates into amorphous silica within an intracellular nano-bioreactor, providing a cost-effective and scalable source of photonic crystal structures. These low cost, biological, nanophotonic structures display versatile morphologies and have many potential applications such as solar cells, drug delivery systems, and selective membranes.

Diatoms suitable for the present invention may be prepared by any suitable method known now to a person of ordinary skill in the art or hereafter developed. In certain working embodiments, diatoms were cultured in flasks containing artificial seawater medium, such as Harrison's artificial seawater medium, with a seeded cell density of about 5×10⁴ cells mL⁻¹ and an initial silicic acid concentration of from about 0.1 mM to about 1 mM, typically from about 0.3 mM to about 0.7 mM, more typically about 0.5 mM. Cultures were incubated for from about 48 hours to about 96 hours, typically about 72 hours, at a suitable temperature of from about 18° C. to about 25° C., typically about 22° C., and were illuminated at light intensities ranging from about 30 to about 150 μM m⁻² sec⁻¹ on a light:dark cycle typically of about 14 hours:10 hours.

Substrates were placed in suitable containers, typically petri dishes, and were covered with a portion of the diatom cell suspension. Suitable substrate materials include any substrate to which the frustules can be attached, such as mica, glass, quartz or metal oxides. In certain working embodiments the substrate material was glass or quartz. Cells were allowed to settle onto the substrates for from about 30 minutes to greater than about 120 minutes, typically from about 45 minutes to about 60 minutes, under from about 30 to about 150 μM m⁻² sec⁻¹ illumination. Liquid was collected and substrates were transferred to clean containers, sealed, for example, with Parafilm, and maintained under illumination for from about 24 hours to greater than about 72 hours, typically about 48 hours, at a suitable relative humidity, such as ambient relative humidity (RH<30%).

Following incubation, the biofilms were soaked in ethanol solutions to stabilize the films and remove soluble organic materials. In certain embodiments the biofilms were soaked for from about 1 hour to about 8 hours, typically from about 3 hours to about 5 hours, in a solution containing ethanol, such as from about 10% ethanol to greater than about 95% ethanol, typically about 70% ethanol. In some embodiments the biofilms were then soaked in about 100% ethanol for from about 1 hour to about 8 hours, typically from about 3 hours to about 5 hours. The biofilms were dried in air and UV-O₃ cleaned for from about 6 hours to about 48 hours, typically for about 12 hours at a suitable temperature, such as from about 60° C. to greater than 100° C., typically about 90° C., with filtered air supplied at 0.5 standard cubic feet per hour (scfh). Diatom frustules may be annealed at a suitable annealing temperature. For certain working embodiments, diatom frustules were annealed at a temperature of from about 200° C. to at least about 600° C., more typically from about 350° C. to about 450° C., in air for an annealing period of from about 30 minutes to greater than 2 hours, typically about 1 hour. Frustules can be annealed for a number of purposes, such as to remove organics and/or in order to improve adhesion to the substrate.

### III. General Embodiments of a Composition Comprising Diatom Frustules

A. Composition Comprising a Metal Film

Certain disclosed embodiments concern a diatom frustule with a metal coating comprising a metal thin film that at least partially covers the frustule surface. The metal film can comprise any suitable metal that will enhance a Raman signal. In certain embodiments the metal is gold and/or silver. The film can be deposited by any suitable method known to a person of ordinary skill in the art, including, but not limited to, thermal evaporation, chemical vapor deposition (CVD), plating, chemical solution deposition, spin coating, physical vapor deposition (PVD), atomic layer deposition, electron beam evaporation, molecular beam epitaxy, sputtering (DC, rf, magnetron), pulsed laser deposition, cathode arc deposition, electrohydrodynamic deposition, atmospheric pressure CVD (APCVD), low-pressure CVD (LPCVD), ultrahigh vacuum CVD (UHVCVD), microwave assisted CVD (MACVD), plasma-enhanced CVD (PECVD), metal-organic CVD (MOCVD), closed space sublimation (CSS), closed spaced vapor transport (CSVT), a liquid deposition, ink-jet deposition, slot die coating, capillary coating, or any combination thereof.

In working embodiments the metal film was deposited by thermal evaporation. Typically, the diatoms were placed in an evaporation system, and evacuated to from about 10⁻⁵ to about 10⁻⁷ Torr. The rate and time of evaporation was selected to make a film of a desired thickness. A typical evaporation rate was about 1.5 Å per second. After deposition of the desired thickness, the sample was heated in an inert atmosphere at an effective temperature and for an effective amount of time to anneal the sample. In working examples the inert atmosphere was an argon atmosphere. The effective temperature was from about 100° C. to greater than about 500° C., typically from about 150° C. to about 300° C. The effective amount of time was from about 1 minute to greater than 60 minutes, typically from about 5 minutes to about 30 minutes.

B. Composition Comprising Nanoparticles

1. Formation of Nanoparticles

Also disclosed herein are embodiments of a composition comprising a diatom frustule and a nanoparticle. As used herein, the term nanoparticle includes nanorods and nanowires unless otherwise specified, and describes a particle having at least one dimension of from about 1 nm to about 1000 nm in size, typically from about 10 nm to 500 nm, more typically from about 50 nm to about 150 nm. In some embodiments the nanoparticles are metal or metal oxide nanoparticles. In certain embodiments the nanoparticles are selected from Au, Ag, Cu, Pt, Pd, Ru, Re, Fe₃O₄ nanoparticles or combinations thereof. In certain working embodiments the nanoparticles were Ag nanoparticles, Au nanoparticles or combinations of Ag and Au nanoparticles.

The nanoparticles can be prepared by any suitable method known to a person of ordinary skill in the art. These include, but are not limited to, photolithography, electron beam lithography, nanosphere lithography, templating, chemical, electrochemical, sonochemical, thermal and photochemical reduction techniques and seed-mediated growth methods. In certain working embodiments, a solution of silver nanoparticles was prepared by the Lee-Meisel method (P. C. Lee and D. Meisel, J. Phys. Chem. 1982, 86, 3391-3395). Briefly, a 1 mM silver nitrate aqueous solution was heated to boiling and sodium citrate (1% by weight) was added. Heating continued for about 1 hour and the color of the solution turned grayish yellow, indicating that the reaction was complete.

2. Linker Attachment

In some embodiments the nanoparticle was attached to the frustule by a linker. Suitable linkers include any molecule that can attach to both the frustule and the nanoparticle. In some embodiments, the linker was an organofunctional alkoxysilane molecule, with a general formula I

(R¹Fn)ₘ(R²O)ₙ(R³)ₚSi I.

With reference to formula I, m is from 1 to 3, n is from 1 to 3, p is from 0 to 2 and m+n+p is 4. R¹ is an alkylene group, i.e. a divalent saturated aliphatic group preferably having from 1 to at least 6 carbon atoms, and more typically 1 to 3 carbon atoms, that are either straight-chained or branched. Exemplary alkylene groups include methylene (—CH₂—), ethylene (—CH₂CH₂—), n-propylene (—CH₂CH₂CH₂—), iso-propylene (—CH₂CH(CH₃)—) or (—CH(CH₃)CH₂—), and the like. R₁ also can be substituted, such as with alkyl groups, halides, heteroatoms etc. R² and R³ are independently aliphatic groups, more typically alkyl groups, i.e. hydrocarbon groups having saturated carbon chains. The chains may be cyclic, branched or unbranched, and typically include from 1 to at least about 10 carbon atoms e.g. methyl, ethyl, n-propyl, isopropyl, n-butyl, iso-butyl, sec-butyl, tert-butyl, n-pentyl, tert-pentyl, neopentyl, isopentyl, sec-pentyl, 3-pentyl, hexyl, heptyl, octyl, nonyl or decyl.

Optionally, R¹, R², or R³ independently may have one or more carbon atoms replaced by a heteroatom, such as oxygen, sulfur or nitrogen. Exemplary groups with a carbon atom replaced by an oxygen include CH₃OCH₂—, —CH₂CH₂OCH₂CH₂—, or (CH₃)₂CH₂OCH₂CH₂—. Fn is a functional group suitable for attaching to a nanoparticle. Exemplary functional groups include, but are not limited to, amino, mercapto, epoxide, carboxylic acid, vinyl, chloro, bromo, iodo, fluoro or hydroxyl.

Exemplary linkers include, but are not limited to, (3-aminopropyl)-triethoxysilane (APTES), (3-aminopropyl)-trimethoxysilane (APS, APTMS), (3-aminopropyl)-diethoxy-methylsilane (APDEMS), (3-aminopropyl)-dimethyl-ethoxysilane (APDMES), (4-aminobutyl)triethoxysilane, (2-aminoethyl)triethoxysilane, aminomethyltriethoxysilane, allyltriethoxysilane, allyltrimethoxysilane, (3-glycidoxypropyl)-dimethyl-ethoxysilane (GPMES), (3-bromopropyl)trimethoxysilane, chloromethyl(methyl)dimethoxysilane, (3-mercaptopropyl)-trimethoxysilane (MPTMS), and (3-mercaptopropyl)-methyl-dimethoxysilane (MPDMS). Combinations of these linkers also may be used.

Optionally, the substrate surface is pretreated to increase the number of available hydroxyl groups on the frustule surface. This pretreatment typically comprised treating the substrate with a solution of H₂O₂/NH₄OH/H₂O in a ratio of about 1:1:5. The substrate was in contact with the solution for an effective time and at an effective temperature to produce the desired effect. Typically, the effective time was for from about 30 minutes to about 2 hours, more typically for about 1 hour. The temperature was from ambient temperature to about 100° C., typically from about 50° C. to about 90° C., more typically about 70° C. After the pretreatment the substrate was washed with water and an alcohol, such as methanol.

The frustules were contacted with a solution of the desired linker in a suitable solvent. Suitable solvents are solvents that will dissolve the linker but not degrade or react with the frustule or substrate. Examples of suitable solvents include, but are not limited to, alcohols, such as methanol, ethanol, propanol or isopropanol, acetonitrile, toluene, water, or combinations thereof. The effective linker solution concentration is from about 1 % by weight to about 50% by weight, more typically from about 5% by weight to about 25% by weight. In certain working embodiments the solution was at a concentration of about 10% by weight. The frustules were in contact with the linker solution for a sufficient time to allow the reaction to proceed to the desired amount, such as to completion. The time is from less than about 1 hour to greater than about 24 hours, typically from about 2 hours to about 12 hours, more typically from about 4 hours to about 6 hours. Following reaction, the frustules were removed from the linker solution and rinsed well. In certain embodiments the frustules were rinsed with an alcohol, such as methanol, and deionized water.

3. Nanoparticle Self-assembly

For certain working embodiments, frustules having a metal coating of nanoparticle coating are made. For frustule-nanoparticle compositions, frustules were then contacted by the nanoparticles in a solution or suspension to allow self-assembly of the nanoparticles on the surface. In certain working embodiments the frustules were contacted by a colloidal suspension of silver particles. The frustules were allowed to contact the nanoparticles for an effective time and at an effective temperature to allow a desired coverage of the frustule surface to occur. The time was typically from about 1 hour to greater than about 24 hours, more typically from about 6 hours to about 18 hours. The temperature was from greater than 0° C. to greater than about 100° C., typically, from about 10° C. to about 50° C., more typically from about 18° C. to about 25° C.

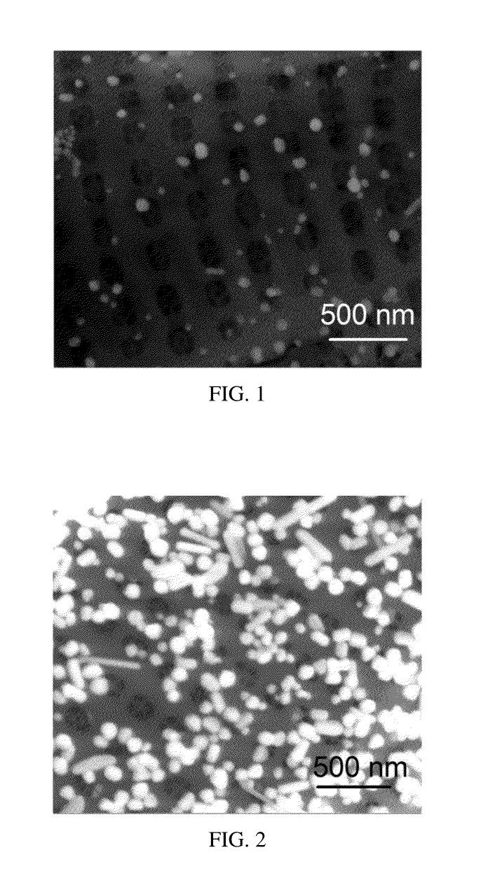

The surface coverage ratio is the percentage of the surface covered by nanoparticles relative to the total available surface area of the frustule. Because the mass of the nanoparticles is large relative to the functional group on the linker, and the bonding process is typically slow, the surface coverage ratio can be precisely controlled by controlling the self-assembly time and/or the dilution of the solution. Typically, the surface coverage ratio varies from about 1% to about 100%, more typically from about 10% to about 80% and in certain embodiments the surface coverage ratio is about 50%. The time and/or dilution required to achieve a particular desired surface coverage ratio for a particular diatom frustule can be determined experimentally. For example, FIGS. 1 and 2 illustrate two APTES pre-treated samples that were immersed in nanoparticles solutions for about 12 hours. The only difference between them was the dilution of the nanoparticle solutions. FIG. 1 shows the nanoparticle coverage when the nanoparticle solution was diluted with water in a ratio of 1:10. FIG. 2 shows the coverage achieved using a nanoparticle solution that was not diluted.

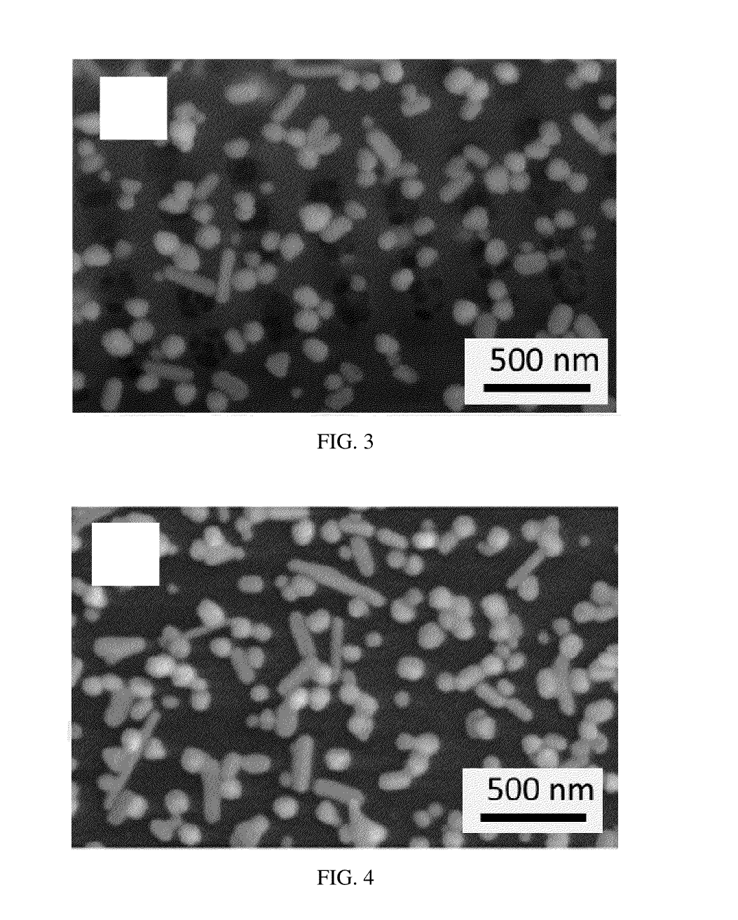

Ag colloids prepared by the Lee-Meisel method yield Ag nanoparticles with a wide range of sizes (such as from about 50 nm to about 150 nm), geometries and aggregation states. Nanoparticles of various aggregation states, including isolated nanoparticles, nanoparticle dimers, trimers, short chains and nanorods, are distributed on the frustule and substrate respectively, as shown in FIG. 3 and FIG. 4.

The nanoparticle size and shape may be less crucial than the size of interparticle gaps in the SERS measurement. As the gap between nanoparticles increases the Raman signal becomes weaker. Sun and Khurgin reported that from calculation results for gap size versus SERS enhancement factors for 25 nm gold nanoparticles, the enhancement factor decays about 2 orders when increasing the gap size from 0 to 10 nm. G. Sun and J. B. Khurgin, “Origin of giant difference between fluorescence, resonance, and nonresonance Raman scattering enhancement by surface plasmons.” Phys. Rev. A, vol. 85, no. 6, pp. 063410-1-063410-8, June 2012. Persons of ordinary skill in the art also may refer to d_{gap}. From Van Duyne group's experimental results, the hot spot is where two nanoparticles are in subnanometer proximity (d_{gap}<1 nm) or have coalesced to form crevices (d_{gap}<0), which maximize SERS enhancements. K. L.Wustholz, A.-I. Henry, J. M. McMahon, R. G. Freeman, N. Valley, M. E. Piotti, M. J. Natan, G. C. Schatz, and R. P. Van Duyne, “Structure-activity relationships in gold nanoparticle dimers and trimers for surface-enhanced Raman spectroscopy.” J Am. Chem. Soc., vol. 132, no. 31, pp.10903-10910, July 2010.

In some areas of the sample shown in FIG. 1 a Raman signal could not be detected. By selecting a suitable combination of self-assembly time and solution dilution, an effective interparticle gap can be selected to produce a desired Raman signal enhancement. The effective interparticle gap is from 0 to about 30 nm, typically from 0 to about 15 nm, more typically from 0 to about 10 nm.

C. Compositions Comprising Antibodies

In some embodiments the composition further comprises an antibody. In certain embodiments the antibody is attached to the organofunctional alkoxysilane by the use of a crosslinking compound. The crosslinking compound is any suitable crosslinking compound known to a person of ordinary skill in the art, which will covalently bond to the functional group of the organofunctional alkoxysilane, and also to the antibody of interest. In some embodiments the cross linking compound is an amine-based crosslinking compound, and in other embodiments it is a thio-based crosslinking compound.

Exemplary crosslinking compounds include, but are not limited to, bis(sulfosuccinimidyl)suberate (BS3), bis(sulfosuccinimidyl)glutarate (BS2G), bis(NHS)PEO₅, bis(2-[succinimidoxycarbonyloxy]ethyl)sulfone, C6-succinimidyl 4-hydrazinonicotinate acetone hydrazine, C6-succinimidyl 4-formylbenzoate, N,N-dicyclohexylcarbodiimide, dimethyl adipimidate.2HCl, disuccinimidyl glutarate, dithiobis(succimidylpropionate), ethylene glycol bis(succinimidylsuccinate), disuccinimidyl suberate, N-(ε-maleimidocaproyloxy)succinimide ester, succinimidyl 4-formylbenzoate, N-hydroxysuccinimidyl-4-azidosalicylic acid, m-maleimidobenzoyl-N-hydroxysuccinimide ester, NHS-PEO₂-maliemide, NHS-PEO₄-maliemide, NHS-PEO₈-maliemide, succinimidyl 4-(p-maleimido-phenyl)butyrate, N-succinimidyl 3-(2-pyridyldithio)propionate, or m-maleimidobenzoyl-N-hydroxysulfosuccinimide ester.

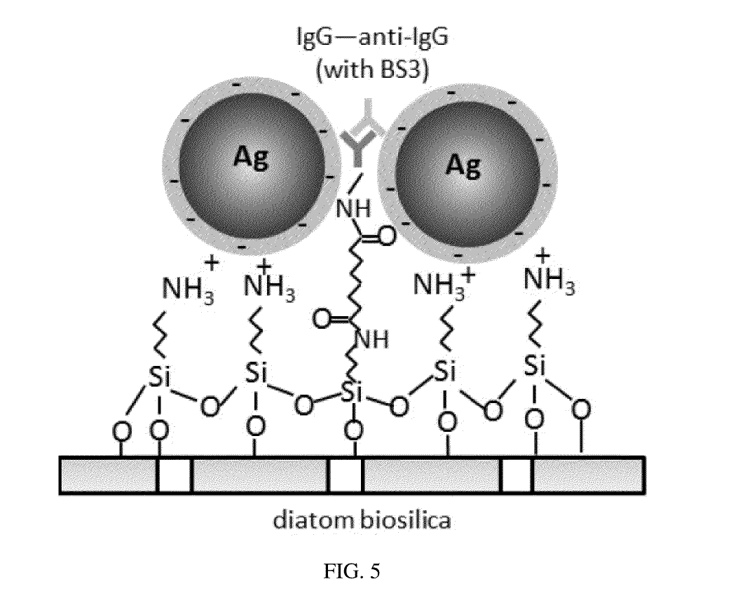

In some embodiments, the crosslinker compound is selected to place the antibody-antigen combination in a position to obtain a desired SERS signal enhancement. In certain embodiments the crosslinker compound is selected such that the crosslinker compound-antibody-antigen combination has a total chain length approximately the same as the radius of the nanoparticles. In particular embodiments, the Ag nanoparticles have a diameter of from about 50 nm to about 80 nm (i.e. a radius of from about 25 nm to about 40 nm), and the BS3 linked antibody-antigen combination has a total chain length of about 30 nm (FIG. 5).

The composition is contacted with a solution of the crosslinking compound in a suitable solvent. The solvent is selected such that the crosslinking compound is soluble in the solvent, but does not react with the solvent. Suitable solvents are known to a person of ordinary skill in the art and include, but are not limited to, toluene, dimethylformamide (DMF), tetrahydrofuran (THF), acetonitrile, alcohols such as methanol, ethanol propanol or isopropanol, dioxane, or DMSO. The mixture of crosslinking compound and diatom composition is allowed to react at an effective temperature and for an effective amount of time to allow the linker molecules not attached to a nanoparticle to react with a crosslinking compound. In some embodiments the effective temperature is from about 0° C. to greater than about 100° C., preferably from about 15° C. to about 50° C., more preferably from about 18° C. to about 26° C. The effective amount of time typically is from greater than 1 minute to about 24 hours, preferably from about 10 minutes to about 18 hours, more preferably from about 30 minutes to about 8 hours. The composition is then removed from the solution by any suitable technique known to a person of ordinary skill in the art, and optionally dried.

The diatom frustule with the crosslinker compound attached is then contacted with the desired antibody, and the mixture is allowed to react at an effective temperature and for an effective amount of time to allow the antibody to react with the crosslinking compound. In some embodiments the effective temperature is from about 0° C. to greater than about 100° C., preferably from about 15° C. to about 50° C., more preferably from about 18° C. to about 26° C. The effective amount of time is from greater than 1 minute to about 48 hours, preferably from about 10 minutes to about 18 hours, more preferably from about 30 minutes to about 8 hours.

In other embodiments, the antibody was attached to the surface of the nanoparticles. In some embodiments the antibodies are adsorbed onto the surface of the nanoparticles through thiol groups, ionic interactions, hydrophobic interactions, hydrophilic interactions or any combination thereof. In certain examples the antibody was attached to the nanoparticle after the nanoparticle was assembled onto the frustule surface. In certain embodiments the frustule was then contacted with an additional blocking compound to block unoccupied sites on the frustule. Suitable blocking compounds are any compound that will effectively block the unoccupied sites while not interfering with the immunoassay. They include, but are not limited to, bovine serum albumin (BSA), rabbit serum albumin, goat serum albumin, nonfat dry milk, casein3 or any combination thereof. In certain working examples BSA was used as a blocking compound.

### IV. Methods of Using the Composition

Also disclosed herein are embodiments of a method of using a composition comprising a diatom frustule and a metal coating. In some embodiments the composition is contacted with a molecule of interest. Typically, the molecule of interest is in a solution or suspension in a solvent. Suitable solvents include, but are not limited to, water, alcohol, such as methanol, ethanol, propanol, or isopropanol, acetonitrile, DMSO, dimethylformamide, toluene, hexanes, ether, a halogenated solvent, such as chloroform, dichloromethane or dichloroethane, acetone, methylethylketone, or any combination thereof. The molecule of interest is added to the composition by any suitable means, such as drop-coating, or by submerging the composition in a solution containing the molecule. In some embodiments, after the addition of the molecule to the composition, the solvent is removed, typically by evaporation.

The composition was then exposed to light comprising a particular wavelength and a spectrum was recorded. The particular wavelength can be a resonance wavelength for the molecule of interest, or a non-resonance wavelength or the light can comprise both resonance and non-resonance wavelengths. SERS with light at a resonance wavelength can result in increased scattering intensities leading to decreased detection limits and measurement times. Alternatively, spectra recorded from SERS using non-resonance wavelengths of light can avoid any complex interference of molecular Raman scattering effects.

Additionally, embodiments of using a composition comprising a diatom frustule, and a metal coating and further comprising an antibody are disclosed. In some embodiments, the frustule composition comprising an antibody was exposed to a target antigen. The antigen could be any suitable compound that will bind to the antibody as a specific binding moiety. In certain embodiments the antibody is selected such that it selectively binds to a particular target antigen that may be in a sample. After exposure to the antigen, the frustule is then contacted with a second nanoparticle functionalized with a second antibody and labeled with a Raman reporter compound. The second nanoparticle may comprise the same material as the first nanoparticle, or it may be a different material. In certain embodiments a gold nanoparticle was assembled onto the frustule and the second nanoparticle comprised silver. In some examples, the antibody on the second nanoparticle is selected such that it will selectively bind to the bound antigen. The Raman reporter compound can be any suitable compound that will provide a signal in Raman spectroscopy. In working embodiments the Raman reporter compound was 5,5′-dithiobis-2-nitrobenzoic acid (DTNB). The composition was then exposed to light comprising a particular wavelength and a Raman spectrum was collected.

### V. Working Examples

### Preparation of a Diatom Frustule Coated with a Silver Thin Film

A 10 nm silver thin film was prepared by thermal evaporation onto diatom frustules using a standard evaporation system (Veeco Instruments, Inc). The chamber was pumped down to 5×10⁻⁶ before evaporation. The evaporation rate was controlled at a rate of 1.5 Å/s. After depositing a 10 nm silver thin film, the sample was annealed in pure argon at 200° C. for 10 minutes.

### Preparation of Diatom Frustules

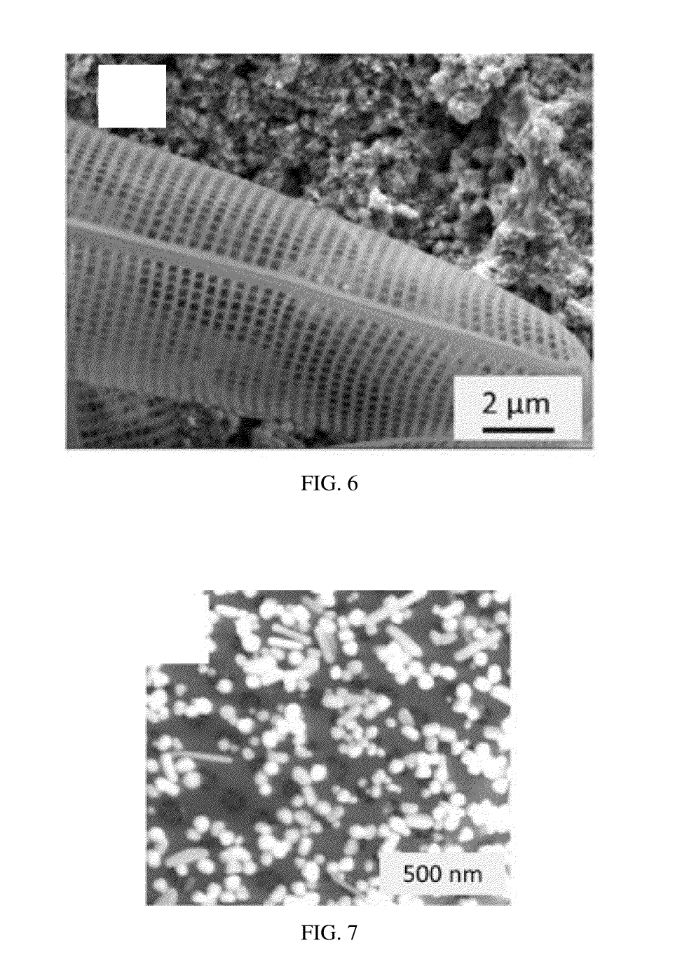

Diatoms were cultured in 500 mL flasks containing 100 mL of Harrison's artificial seawater medium with a seeded cell density of 5×10⁴ cells·mL⁻¹ and an initial silicic acid concentration of 0.5 mM. Cultures were incubated for 72 hours at 22° C., illuminated at 30 μM m⁻² sec⁻¹ on a 14:10 light: dark cycle. Glass substrates (22 mm×22 mm) were placed in 60 mm petri dishes and covered with 15 mL of the diatom cell suspension. Cells were allowed to settle onto the substrates for 45-60 minutes under 150 μM m⁻² sec⁻¹ illumination. Liquid was collected and substrates were transferred to clean 60 mm petri dishes, sealed with Parafilm and maintained under illumination for 48 hours at ambient relative humidity (RH<30%). Residual cells were collected from seeding dishes and combined with collected liquid media for analysis of residual cell counts. Following incubation, biofilms were soaked for four hours in 70% ethanol and four hours in 100% ethanol to stabilize the films and remove soluble organic materials. Biofilms were dried in air and UV-O₃ cleaned for 12 hours at 90° C. with filtered air supplied at 0.5 scfh. The diatom frustules were annealed at 400° C. in air for 1 hour in order to improve adhesion to the glass substrate. A scanning electron microscopy (SEM) image of a representative diatom is presented in FIG. 6.

### Preparation of a Silver Nanoparticle Solution

The aqueous Ag nanoparticle solution was prepared by the conventional Lee Meisel method. Briefly, silver nitrate (0.18%, by weight) solution of 250 mL in deionized water was heated to boiling in the oil bath. Sodium citrate (1%, by weight) solution of 5 mL was added to the silver nitrate solution as soon as the boiling occurred, and heating was continued for an additional 1 hour. The color of the solution turned from colorless into grayish yellow, and then turbid, which indicated the reaction was completed.

### Preparation of a Gold Nanoparticle Solution

An aqueous solution of Au nanoparticles was prepared by the conventional Turkevich method. Briefly, 95 mL of chlorauric acid solution (containing about 5 mg Au) was heated to boiling point and 5 mL of 1% sodium citrate solution was added with stirring. After about 5 minutes the solution darkened. A deep wine red color indicated completion.

### Self-assembly of Silver Nanoparticles on Diatom Frustules

A solution of silver nanoparticles was prepared. The diatom-coated substrates were modified with aminopropyltriethoxyl-silane (APTES) to promote nanoparticle adhesion. The glass substrate coated with diatoms was first cleaned by immersing in a RCA solution (1:1:5 H₂O₂/NH₄OH/H₂O) for 1 hour at 70° C., followed by rinsing with deionized water and rinsing in methanol. High-density hydroxyl groups on glass substrate and diatom surface were created by this pretreatment, which were used to attach the APTES molecules. After cleaning, the diatom samples were immersed in APTES solution (10%, by weight in methanol) for 5 hours, followed by rinsing successively in methanol and deionized water and blowing with high-purity nitrogen. The substrates were then exposed to the Ag colloidal suspension for 12 hours to deposit the Ag nanoparticles onto the frustules and the underlying substrate.

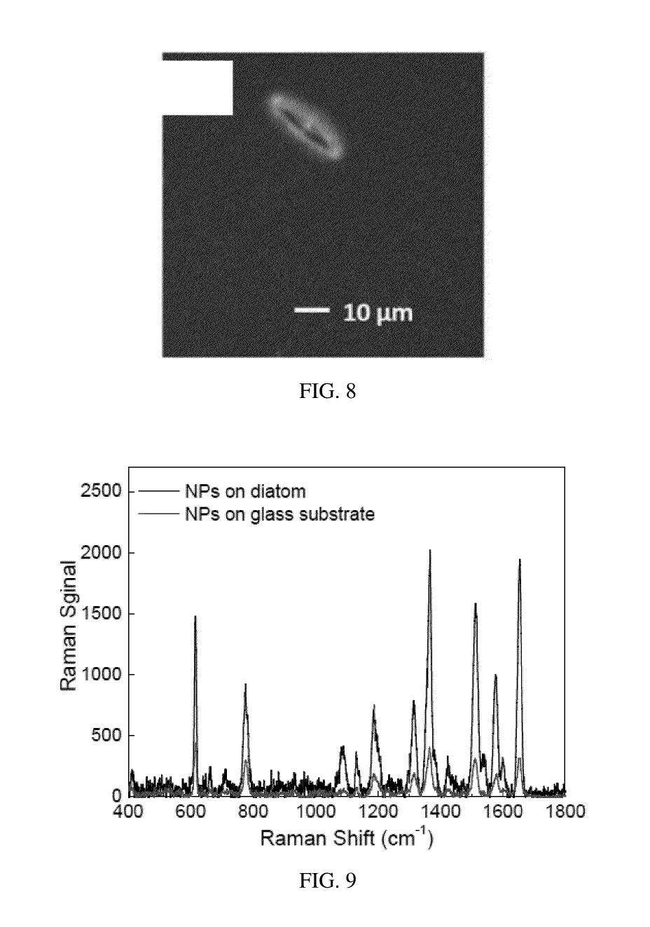

After being removed from the nanoparticle solution, the sample was rinsed with deionized water and dried with high-purity nitrogen. The representative SEM image in FIG. 7 shows the densely assembled Ag nanoparticles on the diatom frustule. Various nanoparticle morphologies were formed, including isolated nanoparticles, nanoparticle dimers, trimers, short chains and nanorods and nanowires on top of the frustule and the glass substrate, which gave multiple plasmonic resonances depending on the aggregation states of the Ag nanoparticles. The average size of the nanoparticles in the preparation was larger than the nanopores, therefore the relative number of nanoparticles located within the diatom pores was small. FIG. 8 shows a dark-field image of self-assembled Ag nanoparticles on diatom frustules demonstrating strong contrast due to optical scattering compared to the self-assembled Ag nanoparticles on the surrounding glass substrate.

### SERS Detection of Rhodmine 6G (R6G) Under Resonance Conditions

For the SERS measurement, R6G molecules in ethanol were drop-coated on the glass substrate with diatom frustules and then evaporated to dryness. Molecular resonance SERS microscopy was performed using a Horiba Jobin-Yvon HR800 confocal Raman spectrometer equipped with a 532 nm diode laser through a notch filter. 532 nm is a molecular resonance wavelength for R6G. During the measurement, the confocal pin-hole size was set at 100 μm. A 50× objective lens (NA=0.75) was used to focus the excitation light to a 2 μm spot totally within a single diatom frustule for each spectrum acquisition. Raman signals were detected by a Synapse charge-coupled device (CCD) detector. FIG. 9 shows the single-point SERS signals measured on the flat glass substrate and on the diatom frustule respectively. SERS signals of the nanoparticles-on-diatom structure show 3.6 to 6.2× enhancement compared with the signals from the nanoparticles-on-glass substrate for major R6G Raman peaks at 614 cm⁻¹, 1368 cm⁻¹, 1511 cm⁻¹, 1578 cm⁻¹ and 1651 cm⁻¹. The additional enhancement of Raman signals was due to the enhanced LSPs due to the presence of the diatom frustule.

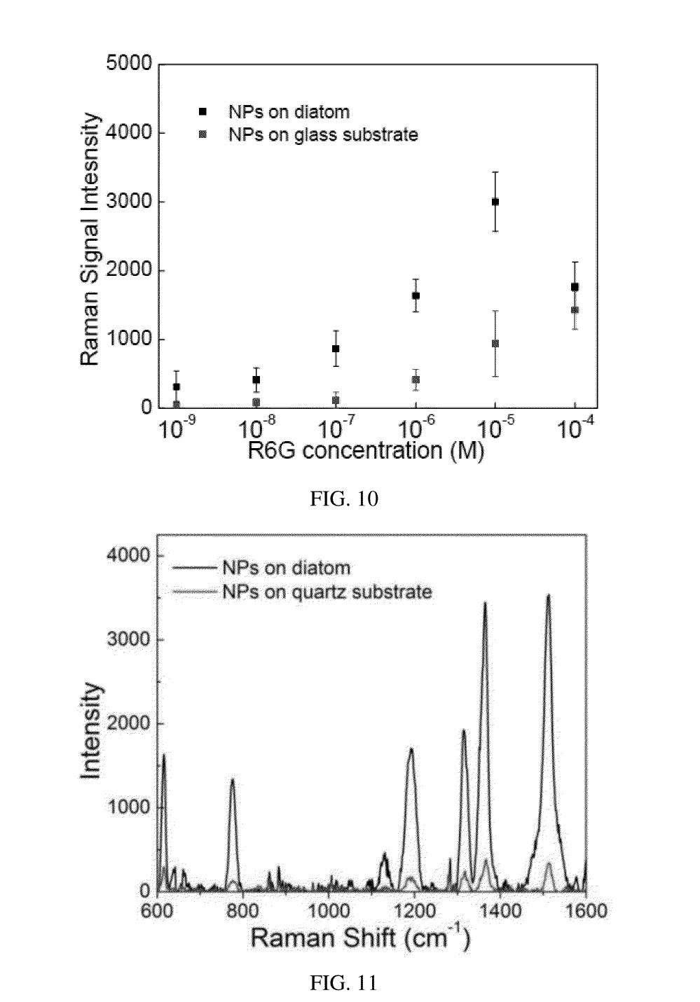

R6G Raman spectra were studied in the concentration range of 10⁻⁹ to 10⁴ M. The Raman band at 1368 cm⁻¹ was used to probe the strength of the SERS of which the intensity were plotted in FIG. 10. The Raman signals increase from 10⁻⁸ M to 10⁻⁵ M after which the Raman intensity decreases at 10⁻⁴ M. The nanoparticle-on-diatom sample showed a 4.1-6.4× enhancement factor compared to the nanoparticle-on-glass, throughout the concentration range of 10⁻⁸ M to 10⁻⁵ M, which are due to the GMRs of the diatom frustule. When further increasing the concentration from 10⁻⁵ M to 10⁻⁴ M, more R6G molecules were attached to the nano-corrugated surface of the frustule, which resulted in a significant increase of florescence baseline. In this case, the florescence signals competed with the SERS signals and degraded the SERS intensity. Therefore, the decreasing of Raman signal intensity was observed after subtracting the florescence baselines when the concentration of R6G molecules was increased above 10⁻⁴ M.

### SERS Detection of R6G Under Non-Resonance Conditions

To obtain SERS detection of R6G under non-resonance conditions, spectra were recorded using 785 nm excitation wavelength. This wavelength does not correspond to the electronic absorption band of R6G. This avoided a complex interference of molecular resonance Raman scattering effects. Due to the fact that the glass substrate displays strong florescence background under the 785 nm wavelength excitation, diatom frustules in aqueous solution were drop-coated on non-florescent quartz substrate for the measurement. The diatom frustules were annealed at 425° C. in air for 1 hour in order to improve the adhesion to the quartz substrate. The fabrication process of self-assembled nanoparticles onto a diatom-coated quartz substrate was identical to the diatom-coated glass substrate as described above with reference to the SERS detection of R6G under resonance conditions. FIG. 11 shows the non-resonance SERS spectra under the 785 nm excitation light from a diode laser. The SERS spectra indicated that the Raman signal intensity was enhanced by 8.7-13.3× on the diatom frustule compared with the flat quartz substrate for the Raman bands of 776 cm⁻¹, 1183 cm⁻¹, 1315 cm⁻¹, 1368 cm⁻¹, and 1511 cm⁻¹. The larger additional enhancement factors from diatom frustules to the R6G Raman signals under non-resonance condition compared with resonance condition is explained by the difference between resonance and non-resonance Raman scattering by surface plasmons. Resonance Raman process is subjected to quenching effect, where an additional loss is introduced for the energy stored in coherent oscillations of molecular dipoles at the excitation wavelengths in the presence of LSP enhancement and GMR enhancement. Compared with resonance Raman process which limits the attainable enhancement, such quenching effect is absent in normal non-resonance Raman scattering.

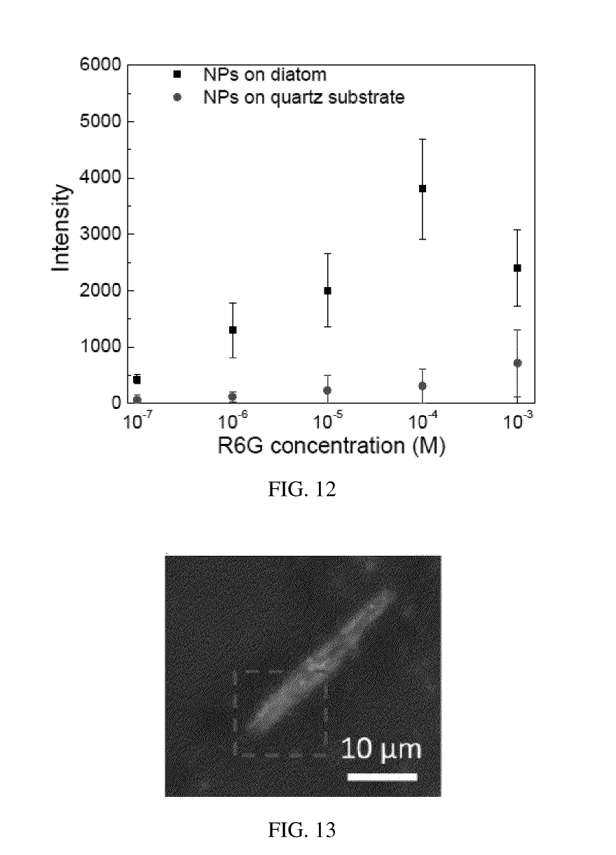

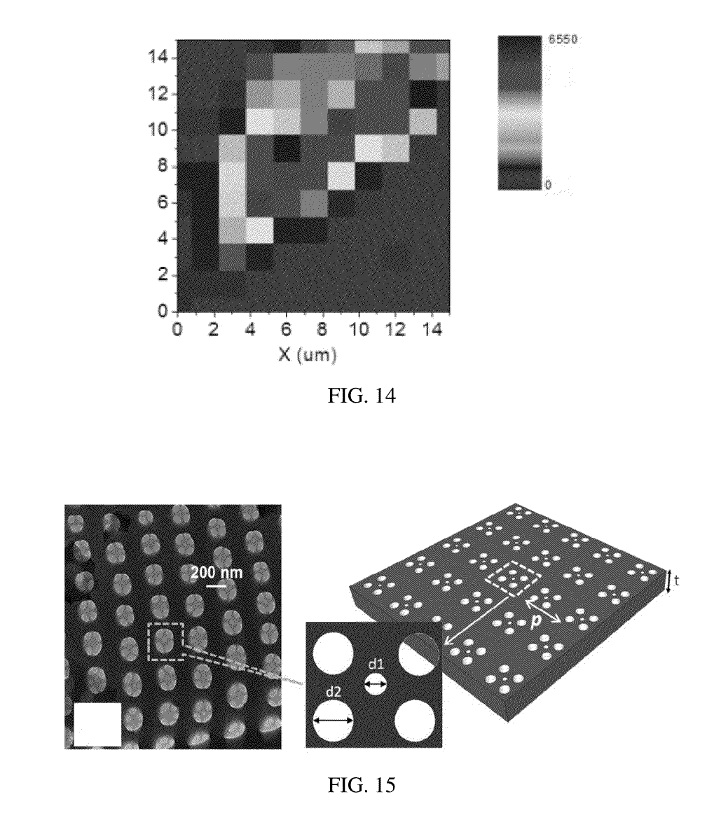

The intensities of the Raman band at 1368 cm⁻¹ were plotted in the concentration ranges from 10⁻⁷ M to 10⁻³ M in FIG. 12. The average enhancement effect to the Raman signals was observed throughout the concentration range of 10⁻⁷ M to 10⁻⁴ M with enhancement factors of 8.9-12.3× between nanoparticles-on-diatom and nanoparticles-on-quartz. For the SERS mapping measurement, an area of 15 μm×15 μm in FIG. 13 was scanned to generate a map in FIG. 14. The average measured SERS signal intensity for the nanoparticles-on-diatom area was 4187±2753, which showed an average signal enhancement of 9.2× compared to quartz substrate whereas the average signal intensity of the nanoparticles-on-quartz area was 457±312. Such enhancement factor for non-resonance SERS is more desirable for biosensing application as fluorescence interference can be avoided compared with resonance SERS.

### Theoretical Modeling and Simulation of Diatom Frustules

### Simulation Model for Diatom Frustules

Diatom frustules can be treated as photonic crystal slabs where the slab thickness will determine the number of slab modes for a given polarization. A model of an ideal diatom photonic crystal slab was constructed from a high resolution scanning electron microcopy (SEM) picture and its guided-mode resonance (GMR) was investigated by using a three-dimensional (3D) frequency domain finite element (FE) solver in Comsol 3.5a. Then, localized plasmonic resonances (LSPs) of metal nanoparticles were studies that were integrated with such highly porous periodic substrates.

A high resolution transmission electron microcopy (TEM) picture of a diatom frustule is shown in FIG. 15, which possess a periodic rectangular lattice air hole structure with sub-100 nm size pores. The schematic diagram of the diatom frustule simulation model shown in FIG. 15 based on this TEM picture. In this investigation, the structure was simplified by using circular air holes instead of deformed air holes to reduce the simulation complexities. The unit cell, as shown in the inset picture of FIG. 15, comprised a center hole with diameter of d1=50 nm and four additional holes with diameters of d2=80 nm on the corners. The slab thickness was fixed at t=120 nm. The periodicity was set to p=450 nm in both directions, which gave a resonant wavelength at 400 nm. Instead of using a periodic boundary condition in the simulation, finite array sizes were considered to determine the actual electric field enhancement.

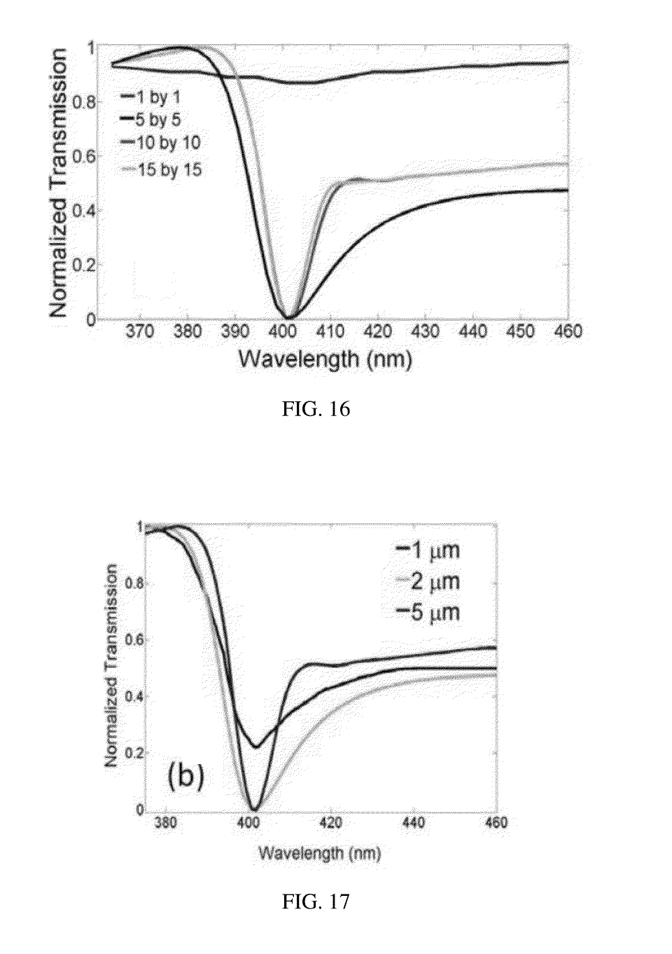

A normally incident Gaussian beam was used, with linear polarization along either direction of the lattice of the periodic pores. The number of unit cells in diatom frustules was set to 1×1, 5×5, 10×10 and 15×15. The normalized transmission was calculated by integrating the power coming out at the bottom boundary of structure and divided by the total power of the Gaussian beam at the input boundary. FIG. 16 shows the transmission spectra of diatom frustules with different numbers of unit cells illuminated by a 5 μm Gaussian beam, which clearly shows that the full width half maximum (FWHM) of GMR became narrower as the array size increased. However, the Q-factor of the GMR was not very high due to the weak index contrast between silica and air. It was also observed that when the array size was greater than 10×10 (15×15 in the simulation), the size effect became negligible as the beam spot (5 μm Gaussian beam in the experiment) was smaller than the diatom size. Therefore, the array size was kept at 10×10 for further investigations to reduce the simulation time. The beam width was also changed to see how the beam divergence affected the GMR properties. FIG. 17 indicates that increasing the beam width sharpened the resonance as more unit cells interacted with the light.

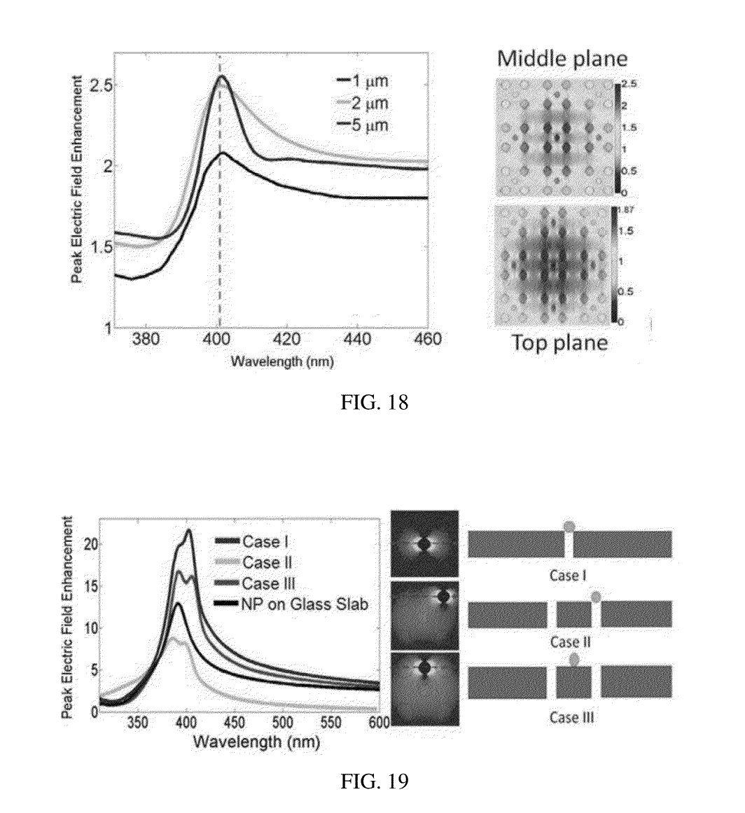

The peak electric field enhancement was obtained by scanning the maximum electric field amplitude throughout the whole volume of the diatom frustule. Again a 10×10 cell frustule was used. By scanning the wavelength, the peak electric field enhancement was simulated. FIG. 18 shows that the peak enhancement occurred at 400 nm, which matched the GMR wavelength. Additionally, the diatom frustule exhibited a nearly flat enhancement profile at wavelengths larger than the resonance wavelength. FIG. 18 also shows the electric field distributions in different planes of the diatom frustule for a 5 gm Gaussian beam. It can be clearly seen that the peak enhancement occurred in the center plane of the frustule, while the electric field was weaker on the top surface.

### LSPs on Diatom Frustules

Integrating nanoparticles into diatom frustules forms two different types of optical couplings: 1) if the nanoparticles are dispensed on top of the surface, they form a weakly coupled system to the frustule, where the total enhancement can be roughly estimated as a product of the individual enhancement factor of the LSPs and GMR; and 2) if the nanoparticles are placed inside the pores, they have a significantly stronger coupling. Because the nanoparticles used for SERS sensing were between 60-100 nm, which was relatively difficult to fit into the pores, only the weak coupling mechanisms were considered in detail.

FIG. 19 shows the maximum enhancement factor from a 50 nm Ag nanoparticle placed on different positions with respect to the center unit cell. The enhancement factor was attributed to the electric field on top of a bare diatom that coupled to the LSPs at the surface of the Ag nanoparticle. This simulation demonstrated that the diatom substrate can induce LSP enhancement by a factor of 2× compared with the glass slab. However, the peak LSP enhancement fell rapidly when the nanoparticle was aligned with the corner holes with larger diameter. Two distinct peaks in the resonance spectrum of the nanoparticle were also observed. This was because the system was weakly coupled and the resonance of the metallic nanoparticle and the grating were detuned from each other to a certain extent. The first peak corresponded to the intrinsic resonance of metallic nanoparticle and the second peak corresponded to the resonance of the GMR of the frustule. The resonance peak of the diatom slightly red shifted to longer wavelength due to the phase retardation induced by the metallic nanoparticle on top of it. Such shift was maximized in case III when the nanoparticle was located on top of the shell (compared with air holes in cases I & II). The intrinsic resonance frequency of the metallic nanoparticle also red shifted due to the change of the surrounding dielectric constants. Because of the pores, the effective dielectric constant of the diatom should be smaller than that of pure silica slab. Such red shift was at maximum when the nanoparticle was placed on the shell (case III) as the surrounding dielectric constant became largest at this location.

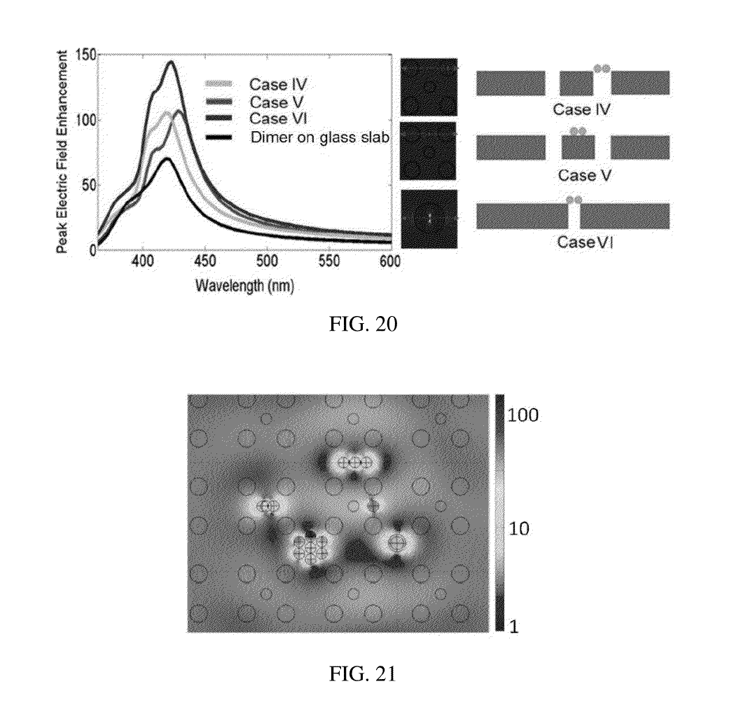

A similar investigation was conducted for a dimer. It can be observed from FIG. 20 that the peak enhancement from a dimer reached up to about 150× when it was aligned with the center hole of the unit cell, while it was only about 70× on a glass substrate. Resonant frequency shift of the diatom due to the presence of dimer became more significant than with a single nanoparticle, because dimer introduced more phase retardation into the system.

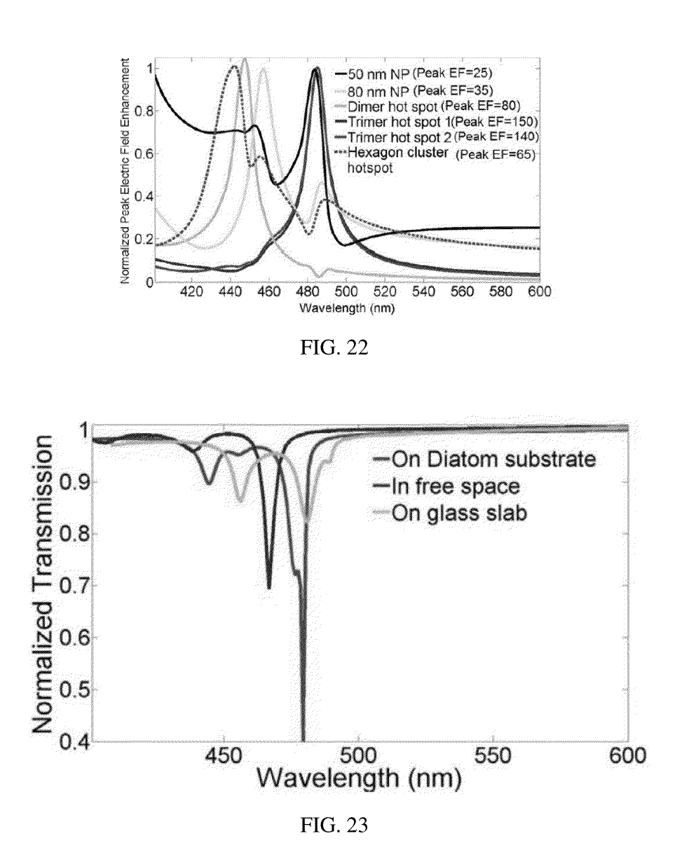

Additionally, an investigation was conducted on more a realistic structure where complex plasmonic nanoparticle morphologies could exist. In this investigation, the diatom substrate was coated by a self-assembly process which formed metal structures of arbitrary shape. The electric field distribution was simulated for an arbitrary assembly of nanostructures consisting of a single nanoparticle (50 nm and 80 nm), a dimer, a trimer and a hexagonal cluster, as illustrated in FIG. 21. The chain structures and the cluster consisted of 50 nm nanoparticles. The gap size between two nanoparticles was fixed at 2 nm. In the analysis, it was observed that the trimer resonance was the dominant one over all other structures. To obtain a clear view into individual resonances, the normalized field enhancement from each structure was plotted in FIG. 22. Several phenomena including blue-shift and red-shift of intrinsic resonances occurred in such a highly interacting system. For example, the resonance wavelength of the 50 nm nanoparticles red-shifted to 480 nm. The resonance spectrum also became completely different than the intrinsic resonance spectrum. However, in this distribution only dimer and trimer structures were dominant and other configurations did not contribute to the transmission spectrum.

To verify the overall transmission, normalized transmission spectra for the given nanoparticle distributions in FIG. 21 were plotted in FIG. 23, which shows nanoparticle distribution in free space, on glass slab, and on diatom substrate. Each spectrum in FIG. 23 only had two distinct peaks, of which the first one at short wavelength was due to the dimer resonance and the second one at long wavelength was due to the trimer resonance. It is clearly visible that the trimer on diatom extinction was nearly 1.75× and 2× stronger compared to the resonance in free space and on glass slab, respectively. This clearly confirmed the potential of a diatom as a SERS substrate.

### Sample Preparation

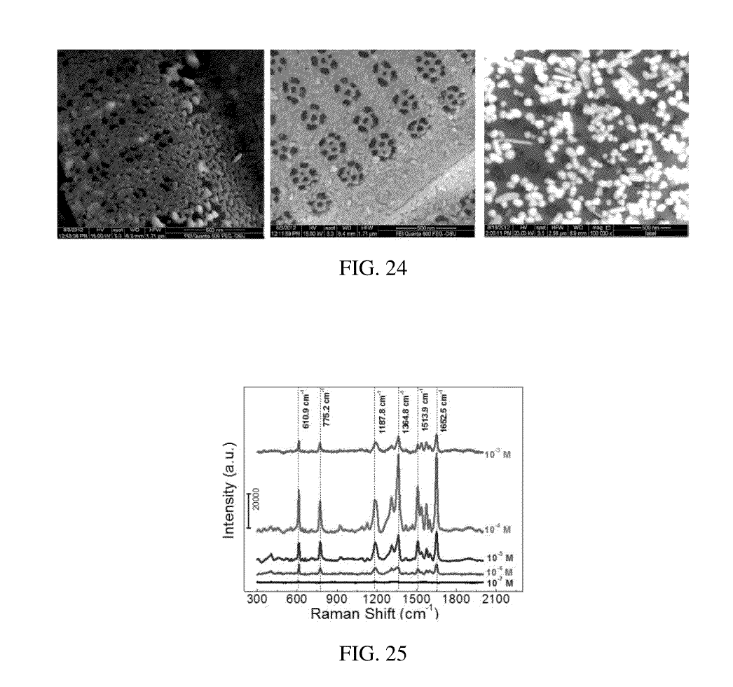

Diatom biofilms of Pinnularia sp. were coated onto glass slides, which were uniformly dispersed on the substrate. The diatom frustules were annealed at 400° C. in air for 1 hour in order to improve adhesion to the glass slides. Three samples were prepared by: 1) thermally evaporating silver films without annealing, 2) thermally evaporating silver films followed by annealing treatment, and 3) self-assembling Ag nanoparticles on the diatom frustules. The 10 nm silver thin film was prepared by thermal evaporation (Veeco Corp.) onto an annealing-treated diatom slide at a rate of 1.5 Å/s. Another sample of silver film was deposited under the same condition, followed by thermal annealing in pure argon at 200° C. for 10 minutes. FIG. 24, left and center show the SEM images for the surface morphology of the thermally evaporated films. Silver islands were formed after thermal evaporation with a very narrow gap size, which became larger after thermal annealing.

The aqueous Ag nanoparticle solution was prepared by conventional Turkevich method. Silver nitrate (0.18%, by weight) solution of 250 mL in deionized water was heated to boiling in the oil bath. Sodium citrate (1%, by weight) solution of 5 mL was added to the silver nitrate solution as soon as the boiling occurred, and heating was continued for an additional 1 hour. The color of the solution turned from colorless into grayish yellow, and then turbid, which indicated the reaction was completed. A monolayer of Ag nanoparticles was formed on a diatom slide by a self-assembly method. The diatom slides was immersed in RCA solution (1:1:5 H₂O₂/NH₄OH/H₂O) for 1 hour at 70° C., followed by rinsing with deionized water and rinsing in methanol. The pretreatment created high density of hydroxyl groups on glass substrate and diatom surface, which were used to link the amino-terminated aminopropyltriethoxylsilane (APTES) molecules. After cleaning, the diatom samples were immersed in an APTES (10%, by weight) solution of methanol for 5 hours, followed by rinsing successively in methanol and deionized water and blowing with high-purity nitrogen. The silver nanoparticles were self-assembled on an aminosilane-pretreated surface by immersing the diatom slides into a colloid suspension for 12 hours. The sample was cleaned with deionized water immediately after removing from the nanoparticle solution, and dried with high-purity nitrogen. The densely self-assembled nanoparticles on diatom surface are shown in FIG. 24, right.

### Experimental Results

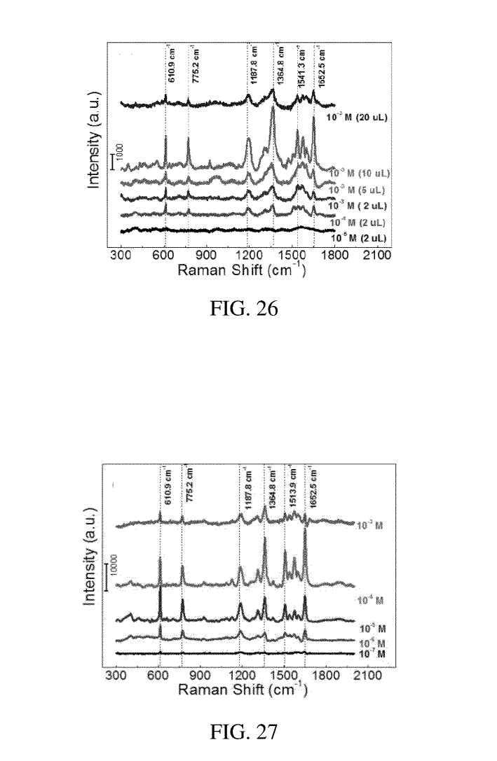

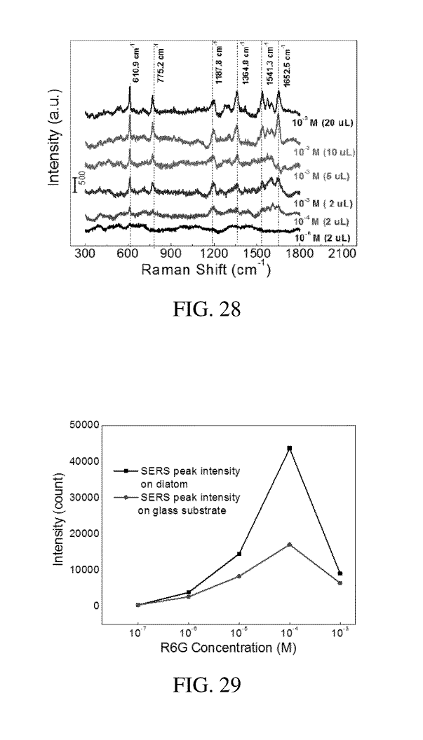

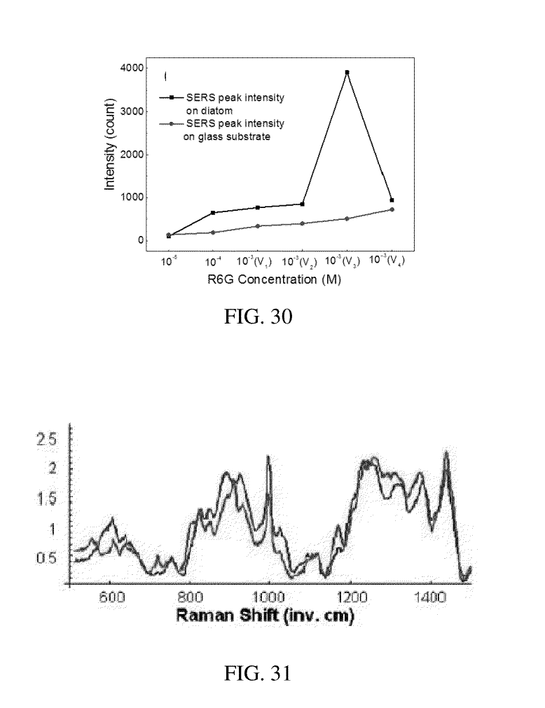

Diatom biosilica were experimentally investigated as a platform for SERS sensing. In the SERS measurement, R6G solutions with concentrations varying from 10⁻⁷ M to 10⁻³ M in ethanol were drop-coated on each sample, which covered the entire sample surface of 5 mm×5 mm. The SERS spectra were obtained using a Horiba Jobin-Yvon LabRAM HR800 spectrometer at 532 nm wavelength, which was equipped with a confocal microscope and a synapse CCD detector. The electric field enhancements of Ag nanoparticles on a flat glass substrate were compared to those on top of diatom frustules. FIG. 25 and FIG. 26 shows the SERS spectra of different concentration R6G observed on self-assembled Ag nanoparticles and on annealing-treated Ag thin films, respectively, in comparison to the Ag nanoparticles and Ag thin film on flat glass substrates, which are shown in FIG. 27 and FIG. 28, respectively. The marked lines correspond to the Raman peaks for R6G. For 10⁻⁷ M to 10⁻³ M concentration, the signal intensities from the SERS-active region on the diatom frustule were about 2× stronger than the glass substrate induced by the localized electric field enhancement from the GMR. FIG. 29 shows the analysis of signal intensities at the peak of 1364.8 cm⁻¹ on diatom frustules and FIG. 30 shows the analysis of signal intensities on bare glass substrates. Signal intensities observed on both diatom and glass dropped down at 10⁻³ M, possibly due to the over absorption of the accumulation of molecules. The signal intensities observed from the diatom were significantly greater below 10⁻³ M, while signals observed on diatom decreased as the number of R6G molecule was increased by repeatedly dropping solution onto the sample.

### Diatom-Based Immunocomplex Formation

The intricate nanostructure of the diatom frustule provides high surface area with free hydroxyl groups (≡Si—OH) that can easily be functionalized to permit tethering of antibodies. IgG-anti IgG will be used as the model system to demonstrate the immune-sensing. Rabbit IgG will be attached to functionalized diatom biosilica in order to detect an increased SERS Raman signal response upon immunocomplex formation with Anti-rabbit IgG. To develop the antibody-functionalized diatom-SERS platform, the process of surface functionalization is based on three steps:

First, the surface silanol (≡Si—OH) groups on the diatom biosilica will be functionalized with amine groups by reaction with 3-amino-propyltrimethoxy-siliane (APS). This APS layer will be used as adsorbing layer for the Ag nanoparticles and for covalent bonding of IgG antibody to the diatom frustule through amine-based crosslinkers such as BS3.

Second, Ag nanoparticles will be self-assembled onto the surface of diatom frustules using APS as the adhesion layer. As the mass of Ag nanoparticles are relatively big and bonding process is relatively slow, the surface coverage ratio by Ag nanoparticles can be precisely controlled by the self-assembling time, varying from 1-80%. The target ratio is 50%, which will balance the presence of Ag nanoparticles and antigen-antibody.

Third, the antibody of interest (rabbit IgG) will be covalently bound to the amine-functionalized diatom biosilica by the BS3 crosslinking reaction. Since these crosslinking reactions are very strong, the antibodies will cover all the remaining frustule surface area that has not been occupied by Ag nanoparticles.

The basic structure of the proposed antibody-functionalized diatom biosilica is schematically shown in FIG. 3. Following functionalization of the diatom biosilica surface and attachment of the antibody and Ag nanoparticles, the device will be ready for testing by examining immuncomplex formation via Raman spectroscopy. In this schematic, it can see that the complementary antigen binding event has a high probability of occurrence in the “hot spots”, especially between the gaps of Ag nanoparticles, where an extremely strong optical field can be concentrated. In principle, for the rabbit-IgG immunocomplex formation, this device can sense the SERS signals of both the IgG and anti-IgG, and even BS3. For example, FIG. 31 shows the SERS spectra of fBAP on a regular Ag colloidal substrate and the same fBAP-modified surface after incubation with fAb solution. The change in SERS spectra is relatively significant. In this project, even stronger signal variation is expected to be observed due to the size effect: the Ag nanoparticles have diameters of from about 50 nm to about 80 nm, while the BS3 linked antibody-antigen has total chain length around 30 nm. This means that the antibody is close to the hot spots, and can obtain higher SERS enhancement factors. It is expected that the diatom-SERS sensors will preferentially amplify the antibody signals.

### Diatom-Based Immuncomplex Formation Using a Reporter Molecule

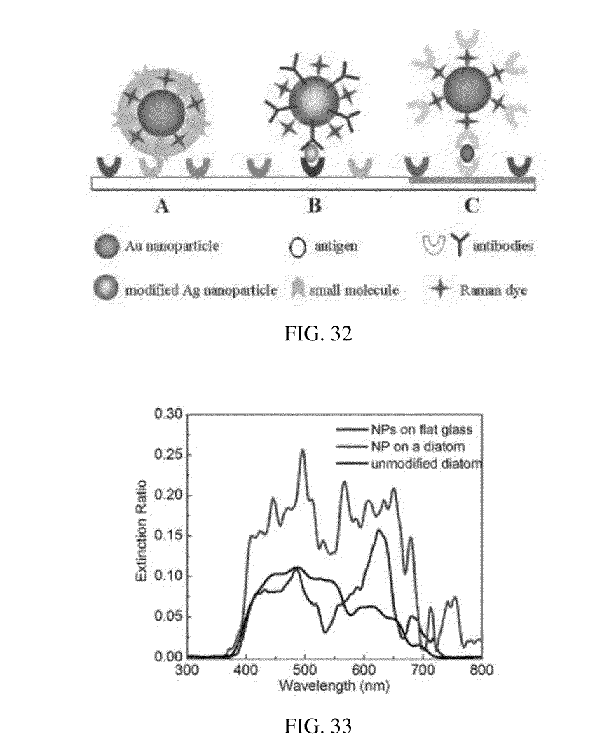

To increase the detection specificity of SERS detection of proteins, Raman reporter molecules will be used when the spectra of antibody-antigen are very similar to each other. For this study IgG and anti-IgG will be used, however the SERS detection will be evaluated by the Raman reporter. 4-mercaptobenzoic acid (MBA) with intensity monitored at 1585 cm-1 shift will be used as the Raman reporter. This large wavenumber shift can avoid overlap with the SERS spectra of most proteins. A common format involves a sandwich assay: antibodies are immobilized on the surface of diatoms, then exposed to antigen, followed by exposure to gold nanoparticles conjugated to antibodies as well as probe molecules. Variations of this format for SERS-based immunoassays are shown in FIG. 32.

### Evaluation of Guided Mode Resonance (GMR) from a Diatom Frustule

In order to confirm the effective enhancement at both 532 nm and 785 nm, a broadband white light source (Intralux 6000) with Vis-NIR (visible to near infrared) output was used as the excitation source. The light was focused by a 40× objective lens (NA=0.65), which was then coupled into a lensed fiber to focus light into the diatom frustules. The sample was mounted on a three-dimensional translation stage. The transmitted light after the sample was collected by a bundled fiber and measured by an Ocean Optics USB4000 spectrometer. The extinction spectra of an unmodified diatom frustule, Ag nanoparticles coated on a flat glass substrate, and Ag nanoparticles coated on a diatom frustule were investigated. A broad, low-Q resonance between 380 nm and 720 nm with a peak extinction at around 480 nm was observed, as shown in FIG. 33. The reduced Q-factor of the observed GMR is believed to be affected by different sources of imperfections including variation of the lattice periodicity, surface roughness, and curvature of the frustules which will cause angular dependence of the incident light. This broadband resonance may help to increase the overall photosynthetic efficiency for diatom over the entire spectrum of visible light.

From the extinction spectra as shown in FIG. 33, the diatom frustule increases the optical extinction ratio of the nanoparticles by ×2 compared to Ag nanoparticles on the glass substrate from 500 nm to 850 nm wavelength.

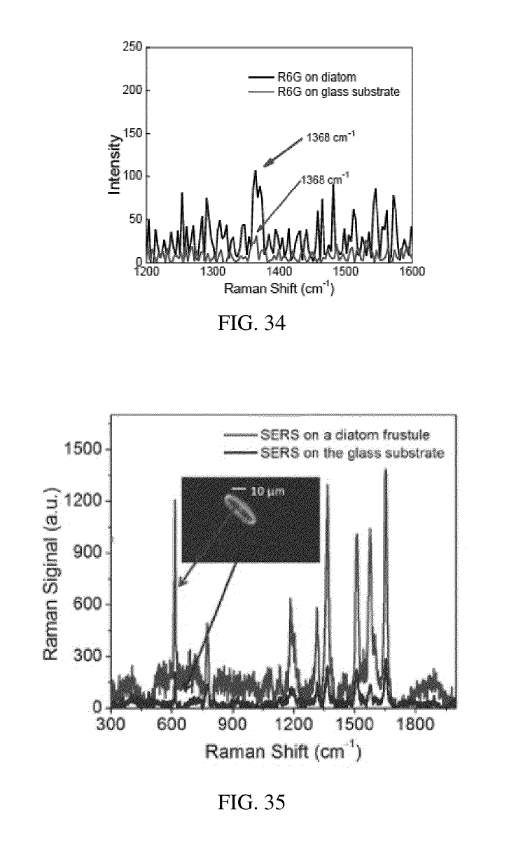

The Raman scattering signals of R6G molecules on the diatom and the glass substrate were also investigated respectively without plasmonic enhancement from nanoparticles in order to confirm the contribution of GMRs from diatom frustules. Raman signals of 5 mM R6G from the diatom frustule and glass substrate are plotted in FIG. 34. The amplitude of the Raman signals is very weak, but it still clearly shows that there is 3.9× enhancement for the Raman peak at 1368 cm⁻¹ from the diatom frustule compared to that from the glass substrate. We attribute this enhancement to the GMRs of the diatom frustules.

The interaction of the Ag nanoparticles with the nanostructured diatom biosilica was also studied. Previous studies have shown that the wavelengths of the LSP resonances at the metal surfaces are determined by the overall geometries and the aggregation states of the Ag nanoparticles. Compared with a single nanoparticle, the aggregation of Ag nanoparticles induces plasmonic extinction at longer wavelengths when individual nanoparticles are in a close-packed assembly and coupled to each other. The frequency and intensity of the plasmon oscillation depend on the degree of aggregation as well as orientation of the individual particles within the aggregate with respect to the polarization direction of the excitation light. For the Ag nanorods, the LSP splits into two modes: one transverse mode with a resonant frequency close to a single nanoparticle (low extinction amplitude), and one longitudinal mode resonant at a much longer wavelength (strong extinction amplitude), which is determined by the nanorod geometry (size and aspect ratio). In the present measurement, the extinction spectrum of Ag nanoparticles observed on the glass substrate showed only two broad resonances: the broad extinction peaks centered at 487 nm and another broad resonance observed at 630 nm. The broadening was due to the excessive radiation damping from large-size nanoparticles, as well as the overlap of multiple resonances of similar aggregation states that have close resonant frequencies. The broad extinction peaks centered at 487 nm were possibly due to the resonances of Ag nanoparticle spherical aggregates (dimers, trimmers, and short chains), whereas the coupling of Ag nanorod dipoles gave rise to the broad resonance observed at 630 nm. However, the extinction peaks due to individual aggregation states could not be clearly identified due to the variations in the nanoparticle dimensions. In the presence of the diatom frustule, two significant differences were observed, as shown in FIG. 33: 1) the diatom frustule increased the optical extinction ratio of the nanoparticle aggregates relative to nanoparticles on flat glass, which was nearly 2× stronger, on average, between 400 nm to 700 nm; and 2) the Q-factors of those LSPs were enhanced by the diatom frustule so that individual LSPs became distinguishable. These differences were attributed to the coupling of the LSPs and the GMR effects of the diatom frustules.

### Verification of the Enhanced LSPs Contributing to Plasmonic Biosensing

To verify whether the enhanced LSPs on diatom biosilica can contribute to plasmonic biosensing, nanoparticles-on-diatom substrates were used as SERS substrates for molecular detection. In the SERS measurement, R6G (20 μL, 1 μM in ethanol), which is widely used as SERS probing molecules, was drop-coated on the sample surface and evaporated to dryness. Samples were analyzed using a Horiba Jobin-Yvon HR800 confocal Raman spectrophotometer with a 532 nm diode laser (1.4 μm spot size), and a Synapse CCD detector. Measurements were taken using a 50× objective lens (NA=0.75), with a 100 μm confocal hole and 1 second integration time. The laser spot size was sufficiently small to be completely contained within a single frustule. The single-point SERS signals measured on the flat glass and on the diatom frustule are plotted FIG. 35. The inset picture shows where the Raman signals were collected for these two measurements. Using the major R6G Raman peaks at 614 cm⁻¹, 776 cm⁻¹, 1368 cm⁻¹, 1511 cm⁻¹ and 1651 cm⁻¹, the Raman signal intensity is enhanced by a factor of 4.79 ±0.8, which clearly indicates enhancement of the LSPs due to the presence of the diatom frustule.

### Immunoassay Using Diatom Frustules

### Preparation of Immunoassay Substrates

SERS immunoassay was performed on Ag self-assembled diatom frustules which were adhered onto glass slides. To immobilize antibody on the silver surfaces, 20 μL goat-anti-mouse IgG (1 mg/mL in PBS, pH 7.36) was pipetted onto each immuno spot. The immobilization was carried out at 4° C. overnight. After rising three times with DI water to remove the free antibody, the remaining active sites on silver surfaces were blocked with 30 μL of 3% BSA in PBS solution for 2 h at room temperature. Then the substrate was rinsed by DI water for three times.

### Preparation of Raman Reporter Labeled Immuno Gold Nanoaggregates

DTNB is a widely used Raman reporter with large Raman cross-section. DTNB molecules can conjugate to the surface of gold nanoparticles via the S-Au bond. In this process, a DTNB molecule was bisected into two TNB molecules, which spontaneously conjugate onto the surface of Au NPs through the thiol group. 20 μL of DTNB solution (1 mM in ethanol) was added into 2 mL of Au NPs and incubated at room temperature overnight. The DTNB functionalized Au NPs were then centrifuged at 9500 rpm for 25 min and redispersed in a mixture of 800 μL DI water and 200 μL PBS solution. The ions in PBS introduced aggregation in DTNB functionalized Au NPs and the color of Au NPs changed from wine red to navy blue.

To further functionalize the Au NPs with antibodies, 20 μL of goat-anti-mouse IgG (1 mg/mL in PBS) was added into the DTNB functionalized Au NPs and incubated on an orbit shaker at a speed of 100 rmp for 2 hours at room temperature. The antibody and DTNB functionalized Au NPs were then centrifuged at 7000 rpm for 15 minutes and redispersed in 1 mL DI water to get rid of the excess antibody. The remaining sites on Au NPs were blocked by BSA by adding 20 μL of 3% BSA in PBS solution and incubated on an orbit shaker at a speed of 100 rmp for 1 hour at room temperature. After centrifuged at 7000 rpm for 15 minutes, the antibody and DTNB functionalized Au NPs were redispersed in 1 mL DI water and stored at 4° C. for further use.

### Immunoassay Protocol

The SERS based immunoassay was carried out according to a standard sandwich protocol of ELISA. Typically, 20 μL antigens (Mouse IgG in PBS) with different concentrations ranging from 1.0×10-3 g/mL to 1.0×10-12 g/mL were pipetted respectively onto each antibody immobilized site on the glass slides and incubated at room temperature. After 2 hours immune recognition, the immunoassay coated with different amount of antigens was rinsed with DI water for 3 times. Afterward, each immuno-site was covered with 20 μL of DTNB and antibody functionalized Au NPs and incubated at room temperature for 1 hour. Finally, the immunoassay was rinsed with DI water for several times and stored at 4° C. for Raman test.

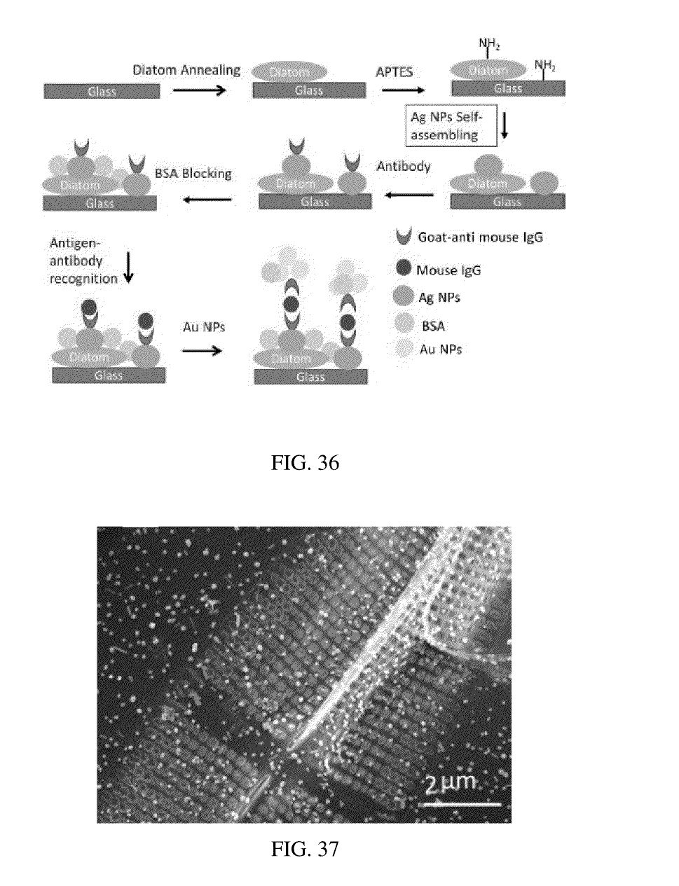

In an exemplary study, diatoms (Pinnularia sp.) were prepared by cultivation, and the diatoms frustules were adhered to glass substrates by low-temperature annealing then functionalized by with aminopropyltriethoxylsilane (APTES) so they would adsorb Ag nanoparticles using the self-assembling method. The immunoassay was designed according to a standard sandwich protocol of enzyme-linked immunosorbent assay (ELISA) as shown in FIG. 36. The model antibody, goat-anti-mouse immunoglobulin G (IgG) was first attached to the surface of Ag nanoparticles. The unoccupied sites were blocked with bovine serum albumin (BSA). The antigen, mouse IgG was then immobilized onto the antibody-functionalized diatom frustules for the specific recognition. To evaluate the specific recognition between the antibody and the antigen, the immunoassay was challenged with a negative control IgG, human IgG. Afterward, the immunoassay was incubated with antibody-functionalized Au nanoparticles, which were initially labeled with Raman reporter, 5,5′-Dithiobis-(2-Nitrobenzoic Acid) (DTNB).

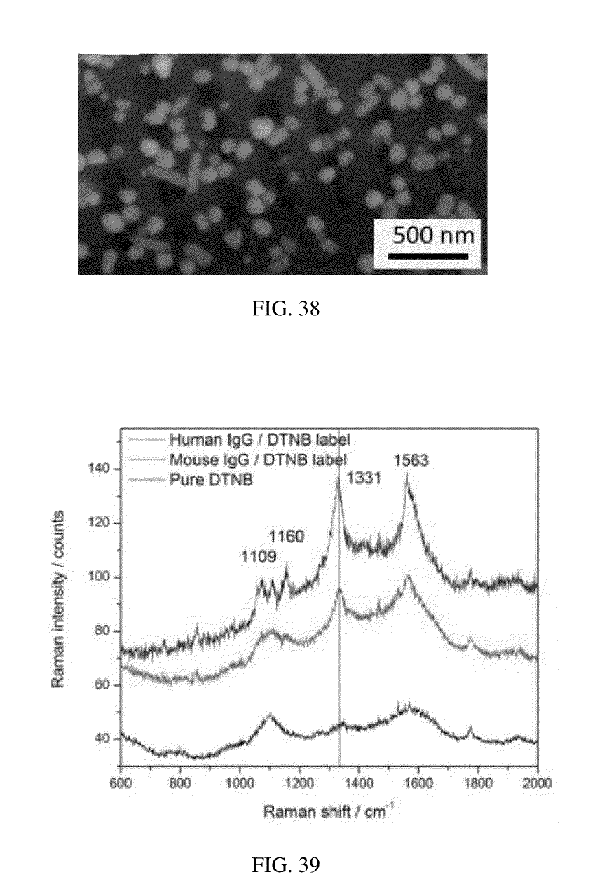

The enhancement of SERS signals was attributed to the interactions between localized surface plasmons (LSPs) of Ag nanoparticles and the guided-mode resonances (GMRs) of diatom frustules. FIG. 37 shows the low-magnification scanning electron microscopy (SEM) of the diatom frustules with self-assembled Ag nanoparticles. It can be seen that the nanoparticle density on the diatom surface and glass substrate is similar. FIG. 38 is the zoomed view of the diatom frustules showing the morphologies of Ag nanoparticles and nanowires (NWs), as well as sub-pores of the frustules with 50-80 nm feature size. These SEM images confirm that the SERS enhancement came from the optical coupling instead of preferential aggregation of metal nanoparticles on the frustules.

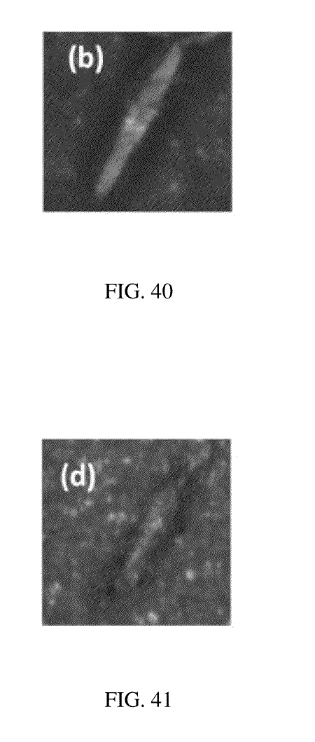

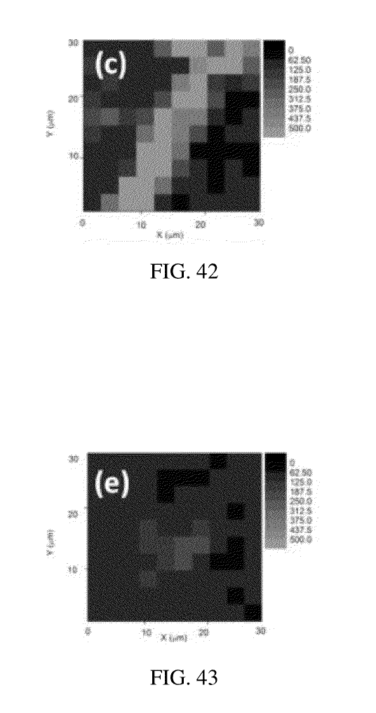

FIG. 39 shows the Raman spectra of 1 mM pure D′TNB, the immunoassay challenged by 1 ng/mL mouse IgG, and the immunoassay challenged by 1 ng/mL human IgG, respectively. All of these signals were obtained on immobilized diatom frustules. From FIG. 39, it was concluded that the SERS spectrum of both mouse-IgG-challenged immunoassay and human-IgG-challenged immune-assay came from the prominent Raman peaks of DTNB. This was because DNB had a much larger Raman scattering cross section than regular antibodies and antigens. In addition, the spectrum width of the Raman peaks from DTNB was much narrower than proteins. The Raman signals of diatom frustules challenged with mouse IgG and human IgG were also mapped, and the optical images are shown in FIG. 40 and FIG. 41, respectively. The results of SERS mapping were plotted according to the intensity of the characteristic Raman band of DTNB at 1331 cm⁻¹. In the positive control, as shown in FIG. 42, the enhancement factors corresponded very well with the morphology of the diatom frustule. The SERS mapping results of the diatom frustule challenged with mouse IgG demonstrated a 6× higher sensitivity than the immunoassay on the nearby glass substrate. While in the negative control results as shown in FIG. 43, the shape of the diatom frustule could not be identified, and the enhancement contrast between the diatom frustule and the nearby glass was only 1.34.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A composition, comprising:
a diatom frustule comprising a surface; and
a metal coating that at least partially covers the surface.

2. The composition according to claim 1, wherein the metal coating comprises metal nanoparticles.

3. The composition according to claim 3, wherein the metal nanoparticles are silver nanoparticles, gold nanoparticles, or a combination thereof.

4. The composition according to claim 3, further comprising a linker.

5. The composition according to claim 4, wherein the linker is an amino silane.

6. The composition according to claim 4, wherein the linker is aminopropyltriethoxy silane or aminopropyltrimethoxy silane.

7. The composition according to claim 1, wherein the surface coverage ratio is greater than about 50%.

8. The composition according to any one of claims 1, further comprising an antibody.

9. The composition according to claim 8, wherein the antibody is attached to a nanoparticle.

10. The composition according to claim 1, further comprising a substrate.

11. A method of making a composition comprising a diatom frustule and a metal coating, the method comprising:
providing a diatom frustule comprising a surface;
contacting the surface with metal to form a metal coating on at least a portion of the surface.

12. The method according to claim 11, wherein the composition has a surface coverage ratio of greater than about 50%.

13. The method according to any one of claims 11, wherein the metal coating comprises metal nanoparticles.

14. The method according to claim 11, further comprising contacting the surface with a linker comprising an amine group.

15. The method according to claim 14, wherein the linker is aminopropyltrimethoxy silane or aminopropyltriethoxy silane.

16. The method according to claim 11, further comprising contacting the composition with an antibody.

17. The method of claim 11, the method comprising:
contacting the surface with a linker to couple the linker to the surface; and
contacting the linker with a composition comprising metal nanoparticles that self-assemble to form a metal coating on at least a portion of the surface.

18. A method of using a composition comprising a diatom frustule and a metal coating, the method comprising:
contacting the composition with a target molecule;
exposing the composition to light comprising a particular wavelength; and
obtaining a spectrum.

19. The method according to claim 18, wherein the particular wavelength of light is a resonance wavelength for the target molecule.

20. The method according to claim 18, wherein the particular wavelength of light is a non-resonance wavelength for the target molecule.

21. The method according to claim 18, wherein the spectrum is a Raman spectrum.

22. The method according to claim 21, wherein the spectrum comprises at least one signal, and the signal has at least a 2-fold enhancement compared to a signal from a spectrum obtained without a diatom frustule.

23. A sensor, comprising:
a diatom frustule comprising a surface;
a linker connecting the surface to a first metal nanoparticle; and
a first antibody.

24. The sensor according to claim 23, further comprising a second metal nanoparticle and a second antibody.
